SEQUENCE LISTING (20 sequences)

SEQ ID NO: 1 - DNA, 3421 bp, Artificial
  Cassette (1)..(3421): Jerusalem artichoke SST expression cassette
gtcgacgatatcggatcctctagacccgggaagcttgagacaggagataaaagtagaaactggatacaacactttgtaacatagtgacactctctccttcctttcttttaccttagaactatacatagaatctacattcaataaaaatacagtaggtacgccgagagatttaaaatgagtaagctaacataccaactaaggccctgtttgtttcggattataatctctccagattatataatccagcgtaaataattcagcagataaacaaacacctaaattatatgttcagattatataatctatagcggagattatgataatctcgtaatctcctaagagtagcttatttgagatttttttggcaaaagacccactaccgttatgtaaatagaattacaatatatgacatccttctttcttcacctcaaataaacaaacaagggtactgttgctttatgaataatctacatttatataatctagactaacaaacaactacatatagattataatacgtctagattataatctagattatataatttaaattatagtcgatattatataatctataagctaaaacaaatagcccctaattattaggctattagttgttaggctatttaaatctaagcgtaaaacgaactaatagcttattagttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagctagccaacccaacacacacccgagcatatcacagtgacagacactacaccatggtggtttcatccaccaccaccacccctctcattctccatgatgaccctgaaaacctcccagaactcaccggttctccgacaactcgtcgtctatccatcgcaaaagtgctttcggggatccttgtttcggttctggttataggtgctcttgttgctttaatcaacaaccaaacatatgaatccccctcggccaccacattcgtaactcagttgccaaatattgatctgaagcgggttccaggaaagttggattcgagtgctgaggttgaatggcaacgatccacttatcattttcaacccgacaaaaatttcattagcgatcctgatggcccaatgtatcacatgggatggtatcatctattttatcagtacaaccctcaatctgccatctggggaaacatcacatggggccactcggtatcgaaagacatgatcaactggttccatctccctttcgccatggttcctgaccattggtacgacatcgaaggtgtcatgacgggttcggctacagtcctccctaatggtcaaatcatcatgctttactcgggcaacgcgtatgatctctcccaagtacaatgcttggcgtacgctgtcaactcgtcggatccacttcttatagagtggaaaaaatatgaaggtaaccctgtcttactcccaccaccaggagtaggctacaaggactttcgggacccatccacattgtggtcgggccctgatggtgaatatagaatggtaatggggtccaagcacaacgagactattggctgtgctttgatttaccataccactaattttacgcattttgaattgaaagaggaggtgcttcatgcagtcccacatactggtatgtgggaatgtgttgatctttacccggtgtccaccgtacacacaaacgggctggacatggtggataacgggccaaatgttaagtacgtgttgaaacaaagtggggatgaagatcgccatgattggtatgcaattggaagttacgatatagtgaatgataagtggtacccagatgacccggaaaatgatgtgggtatcggattaagatatgattttggaaaattttatgcgtccaagacgttttatgaccaacataagaagaggagagtcctttggggctatgttggagaaaccgatccccaaaagtatgacctttccaagggatgggctaacattttgaatattccaaggaccgtcgttttggacctcgaaactaaaaccaatttgattcaatggccaatcgaggaaaccgaaaaccttaggtcgaaaaagtatgatgaatttaaagacgtcgaacttcgacccggggcactcgttccccttgagataggcacagccacacagttggatatagttgcgacattcgaaatcgaccaaaagatgttggaatcaacgctagaggccgatgttctattcaattgcacgactagtgaaggctcggttgcaaggagtgtgttgggaccgtttggtgtggtggttctagccgatgcccagcgctccgaacaacttcctgtatacttctatatcgcaaaagatattgatggaacctcacgaacttatttttgtgccgacgaaacaagatcatccaaggatgtaagcgtagggaaatgggtgtacggaagcagtgttcctgtcctccctggcgaaaagtacaatatgaggttattggtggatcattcgatagtagagggatttgcacaaaacgggagaaccgtggtgacatcaagagtgtatccaacaaaggcgatctacaacgctgcgaaggtgtttttgttcaacaacgcgactggaatcagtgtgaaggcgtcgatcaagatctggaagatgggggaagcagaactcaatcctttccctcttcctgggtggactttcgaactttgatggttatattttggaccctatatatgtgttattatcatgatggttatattttggaccctatatatgtgttattatcatgaagcataagtttggactggagggggtattattgtaattttatatgcatgttctattacttgatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagcttcagctgc

SEQ ID NO: 2 - DNA, 3245 bp, Artificial
  Cassette (1)..(3245): Jerusalem Artichoke FFT expression cassette
actagtggatcccccgggaagcttgagacaggagataaaagtagaaactggatacaacactttgtaacatagtgacactctctccttcctttcttttaccttagaactatacatagaatctacattcaataaaaatacagtaggtacgccgagagatttaaaatgagtaagctaacataccaactaaggccctgtttgtttcggattataatctctccagattatataatccagcgtaaataattcagcagataaacaaacacctaaattatatgttcagattatataatctatagcggagattatgataatctcgtaatctcctaagagtagcttatttgagatttttttggcaaaagacccactaccgttatgtaaatagaattacaatatatgacatccttctttcttcacctcaaataaacaaacaagggtactgttgctttatgaataatctacatttatataatctagactaacaaacaactacatatagattataatacgtctagattataatctagattatataatttaaattatagtcgatattatataatctataagctaaaacaaatagcccctaattattaggctattagttgttaggctatttaaatctaagcgtaaaacgaactaatagcttattagttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagctagccaacccaacacacacccgagcatatcacagtgacagacactacaccatggaaacccctgaaccctttacagaccttgaacatgaaccccacacacccctactggaccaccaccacaacccaccaccacaaaccaccacaaaacctttgttcaccagggttgtgtccggtgtcacctttgttttattcttctttgctttcgctatcgtattcattgttctcaaccaacagaattcttctgttcgtatcgtcaccaattcggagaaatcttttataaggtattcgcagaccgatcgcttgtcgtgggaacggaccgcttttcattttcagcctgccaagaattttatttacgatccagatggtcagttgtttcacatgggctggtaccatatgttctatcaatacaacccatacgcaccggtttggggcaatatgtcatggggtcactcagtgtccaaagacatgatcaactggtacgagctgccagtcgctatggtcccgaccgaatggtatgatatcgagggcgtcttatccgggtctaccacggtccttccaaacggtcagatctttgcattgtatactgggaacgctaatgatttttcccaattacaatgcaaagctgtacccgtaaacttatctgacccgcttcttattgagtgggtcaagtatgaggataacccaatcctgtacactccaccagggattgggttaaaggactatcgggacccgtcaacagtctggacaggtcccgatggaaagcataggatgatcatgggaactaaacgtggcaatacaggcatggtacttgtttactataccactgattacacgaactacgagttgttggatgagccgttgcactctgttcccaacaccgatatgtgggaatgcgtcgacttttacccggtttcgttaaccaatgatagtgcacttgatatggcggcctatgggtcgggtatcaaacacgttattaaagaaagttgggagggacatggaatggattggtattcaatcgggacatatgacgcgataaatgataaatggactcccgataacccggaactagatgtcggtatcgggttacggtgcgattacgggaggttttttgcatcaaagagtctttatgacccattgaagaaaaggaggatcacttggggttatgttggagaatcagatagtgctgatcaggacctctctagaggatgggctactgtttataatgttggaagaacaattgtactagatagaaagaccgggacccatttacttcattggcccgttgaggaagtcgagagtttgagatacaacggtcaggagtttaaagagatcaagctagagcccggttcaatcattccactcgacataggcacggctacacagttggacatagttgcaacatttgaggtggatcaagcagcgttgaacgcgacaagtgaaaccgatgatatttatggttgcaccactagcttaggtgcagcccaaaggggaagtttgggaccatttggtcttgcggttctagccgatggaaccctttctgagttaactccggtttatttctatatagctaaaaaggcagatggaggtgtgtcgacacatttttgtaccgataagctaaggtcatcactagattatgatggggagagagtggtgtatgggggcactgttcctgtgttagatgatgaagaactcacaatgaggctattggtggatcattcgatagtggaggggtttgcgcaaggaggaaggacggttataacatcaagggcgtatccaacaaaagcgatatacgaacaagcgaagctgttcttgttcaacaacgccacaggtacgagtgtgaaagcatctctcaagatttggcaaatggcttctgcaccaattcatcaataccctttttaattaccggctatcgggatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagcttatcgataccgtcgacc

SEQ ID NO: 3 - DNA, 2116 bp, Artificial
  cassette (1)..(2116): bar gene expression cassette
agcttgcatgcctgcaggtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcaggactctagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatccgatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatggcctttgattcagtaggaactactttcttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttgatgagacctgccgcgtaggctagaggatccccggga

SEQ ID NO: 4 - DNA, 25 bp, Artificial
  primer (1)..(25): oligonucleotide primer
ttcgtaactcagttgccaaatattg

SEQ ID NO: 5 - DNA, 22 bp, Artificial
  primer (1)..(22): Oligonucleotide primer
ccagcccgtttgtgtgtacggt

SEQ ID NO: 6 - DNA, 21 bp, Artificial
  primer (1)..(21): Oligonucleotide primer
gttcgtatcgtcaccaattcg

SEQ ID NO: 7 - DNA, 21 bp, Artificial
  primer (1)..(21): Oligonucleotide primer
gtgcactatcattggttaacg

SEQ ID NO: 8 - DNA, 35 bp, Artificial
  primer (1)..(35): Oligonucleotide primer
aagcttgcggccgcgccatggcttcmtchaccacc

SEQ ID NO: 9 - DNA, 41 bp, Artificial
  primer (1)..(41): Oligonucleotide primer
aagcttctcgaggcggccgctcaagaagtccacccagtaac

SEQ ID NO: 10 - DNA, 2034 bp, Parthenium argentatum
atggtggtttcatccaccaccaccacccctctcattctccatgatgaccctgaaaacctcccagaactcaccggttctccgacaactcgtcgtctatccatcgcaaaagtgctttcggggatccttgtttcggttctggttataggtgctcttgttgctttaatcaacaaccaaacatatgaatccccctcggccaccacattcgtaactcagttgccaaatattgatctgaagcgggttccaggaaagttggattcgagtgctgaggttgaatggcaacgatccacttatcattttcaacccgacaaaaatttcattagcgatcctgatggcccaatgtatcacatgggatggtatcatctattttatcagtacaaccctcaatctgccatctggggcaacatcacatggggccactcggtatcgaaagacatgatcaactggttccatctccctttcgccatggttcctgaccattggtacgacatcgaaggtgtcatgacgggttcggctacagtcctccctaatggtcaaatcatcatgctttactcgggcaacgcgtatgatctctcccaagtacaatgcttggcgtacgctgtcaactcgtcggatccacttcttatagagtggaaaaaatatgaaggtaaccctgtcttactcccaccaccaggagtaggctacaaggactttcgggacccatccacattgtggtcgggccctgatggtgaatatagaatggtaatggggtccaagcacaacgagactattggctgtgctttgatttaccataccactaattttacgcattttgaattgaaagaggaggtgcttcatgcagtcccacatactggtatgtgggaatgtgttgatctttacccggtgtccaccgtacacacaaacgggctggacatggtggataacgggccaaatgttaagtacgtgttgaaacaaagtggggatgaagatcgccatgattggtatgcaattggaagttacgatatagtgaatgataagtggtacccagatgacccggaaaatgatgtgggtatcggattaagatatgattttggaaaattttatgcgtccaagacgttttatgaccaacataagaagaggagagtcctttggggctatgttggagaaaccgatccccaaaagtatgacctttcaaagggatgggctaacattttgaatattccaaggaccgtcgttttggacctcgaaactaaaaccaatttgattcaatggccaatcgaggaaaccgaaaaccttaggtcgaaaaagtatgatgaatttaaagacgtcgagcttcgacccggggcactcgttccccttgagataggcacagccacacagttggatatagttgcgacattcgaaatcgaccaaaagatgttggaatcaacgctagaggccgatgttctattcaattgcacgactagtgaaggctcggttgcaaggagtgtgttgggaccgtttggtgtggtggttctagccgatgcccagcgctccgaacaacttcctgtatacttctatatcgcaaaagatattgatggaacctcacgaacttatttttgtgccgacgaaacaagatcatccaaggatgtaagcgtagggaaatgggtgtacggaagcagtgttcctgtcctcccaggcgaaaagtacaatatgaggttattggtggatcattcgatagtagagggatttgcacaaaacgggagaaccgtggtgacatcaagagtgtatccaacaaaggcgatctacaacgctgcgaaggtgtttttgttcaacaacgcgactggaatcagtgtgaaggcgtcgatcaagatctggaagatgggggaagcagaactcaatcctttccctcttcctgggtggactttcgaactttgatggttatattttggaccctatatatgtgttattatcatgatggttatattttggaccctatatatgtgttattatcatgaagcataagtttggactggagggggtattattgtaattttatatgcatgttctattacttga

SEQ ID NO: 11 - DNA, 38 bp, Artificial
  primer (1)..(38): Oligonucleotide primer
aagcttgcggccgcaccatggcaacccctgaacaaccc

SEQ ID NO: 12 - DNA, 41 bp, Artificial
  primer (1)..(41): Oligonucleotide primer
aagcttctcgaggcggccgcctaattaaactcgtattgatg

SEQ ID NO: 13 - DNA, 1862 bp, Parthenium argentatum
atggaaacccctgaaccctttacagaccttgaacatgaaccccacacacccctactggaccaccaccacaacccaccaccacaaaccaccacaaaacctttgttcaccagggttgtgtccggtgtcacctttgttttattcttctttggtttcgctatcgtattcattgttctcaaccaacagaattcttctgttcgtatcgtcaccaattcggagaaatcttttataaggtattcgcagaccgatcgcttgtcgtgggaacggaccgcttttcattttcagcctgccaagaattttatttacgatccagatggtcagttgtttcacatgggctggtaccatatgttctatcaatacaacccatacgcaccggtttggggcaatatgtcatggggtcactcagtgtccaaagacatgatcaactggtacgagctgccagtcgctatggtcccgaccgaatggtatgatatcgagggcgtcttatccgggtctaccacggtccttccaaacggtcagatctttgcattgtatactgggaacgctaatgatttttcccaattacaatgcaaagctgtacccgtaaacttatctgacccgcttcttattgagtgggtcaagtatgaggataacccaatcctgtacactccaccagggattgggttaaaggactatcgggacccgtcaacagtctggacaggtcccgatggaaagcataggatgatcatgggaactaaacgtggcaatacaggcatggtacttgtttactataccactgattacacgaactacgagttgttggatgagccgttgcactctgttcccaacaccgatatgtgggaatgcgtcgacttttacccggtttcgttaaccaatgatagtgcacttgatatggcggcctatgggtcgggtatcaaacacgttattaaagaaagttgggagggacatggaatggattggtattcaatcgggacatatgacgcgataaatgataaatggactcccgataacccggaactagatgtcggtatcgggttacggtgcgattacgggaggttttttgcatcaaagagtctttatgacccattgaagaaaaggaggatcacttggggttatgttggagaatcagatagtgctgatcaggacctctctagaggatgggctactgtttataatgttggaagaacaattgtactagatagaaagaccgggacccatttacttcattggcccgttgaggaagtcgagagtttgagatacaacggtcaggagtttaaagagatcaagctagagcccggttcaatcattccactcgacataggcacggctacacagttggacatagttgcaacatttgaggtggatcaagcagcgttgaacgcgacaagtgaaaccgatgatatttatggttgcaccactagcttaggtgcagcccaaaggggaagtttgggaccatttggtcttgcggttctagccgatggaaccctttctgagttaactccggtttatttctatatagctaaaaaggcagatggaggtgtgtcgacacatttttgtaccgataagctaaggtcatcactagattatgatggggagagagtggtgtatgggggcactgttcctgtgttagatgatgaagaactcacaatgaggctattggtggatcattcgatagtggaggggtttgcgcaaggaggaaggacggttataacatcaagggcgtatccaacaaaagcgatatacgaacaagcgaagctgttcttgttcaacaacgccacaggtacgagtgtgaaagcatctctcaagatttggcaaatggcttctgcaccaattcatcaataccctttttaattaccggctatcgg

SEQ ID NO: 14 - DNA, 6742 bp, Expression vector pJMS02
  misc_feature (5830)..(5830): n = A, C, G, or T
ggccgcgccatggcttcatctaccaccacctcccctctcattctccacgatgatcctgaaaacctccaggaacccaccggatttacgggggttcgtcgtccatccatcgcaaaagcgctttgcgtaacccttgtttcggttatggtaatctgtggtctggttgctgtaatcagcaaccagacacaggtaccacaagtagccaacagccatcaaggtgccgccaccacattcacaactcagttgccaaaaatagatatgaaacgggttccgggagagttggattcgggtgctgatgtccaatggcaacgctccgcttatcattttcaacctgacaaaaactacattagtgatcctgatggcccaatgtatcacatgggatggtaccatctattttatcagtacaacccagaatctgccatatggggcaacatcacatggggtcactccgtatccaaagacatgatcaactggttccatctccctttcgccatggttccggaccattggtacgacatcgaaggcgtcatgacaggttccgccacagtcctcccaaacggtgagatcatcatgctttacacgggcaatgcgtacgatctctcccaagtacaatgcttagcgtacgcagtcaactcatcagatccacttcttatagagtggaaaaaatacgaaggcaacccggttttattgccgccgccaggggtgggttacaaggattttcgggacccatctacattgtggctgggccccgatggtgaatatagaatggtaatggggtccaagcacaacgagactattggttgtgctttgatttaccataccactaattttacgcattttgaattgaatgaggaggtgcttcatgcggtcccacatactggtatgtgggaatgcgttgatctttatccggtatccaccacacacacaaacgggttggacatggtggataatgggccaaatgtaaaatacgtgttgaaacaaagtggggatgaagatcgccatgattggtatgcgattggaagttatgattgggtgaatgataagtggtacccggatgacccggaaaacgatgtgggtatcgggttaagatacgattacggaaagttttatgcgtccaagacgttttatgaccaacataagaaaaggagggtcctttggggctatgttggagaaaccgatcccgaaaagtatgaccttacaaagggatgggctaacatattgaatattccaaggaccgtcgttttggacacgaaaactaaaaccaatttgattcaatggccaattgaggaaaccgaaaaacttaggtcgaaaaagtatgataaatttgtagatgtggagcttcgacccgggtcactcattcccctcgagataggtacagccacacagttggatatagttgcgacattcgaagttgatcaaatgatgttggaatcaacgctagaagccgatgttctattcaactgcacgactagtgttggctcagttggaaggggcgtgttgggaccgtttggtgtggtggttctagctgatgcccagcgcaccgaacaacttcctgtgtatttctatattgcaaaagataccgacgggacgtcaagaacctacttttgtgctgatgaaacaagatcatccaaggatgtagacgtggggaaatgggtgtatggaagcagtgttcctgtcctccctaacgaaaagtacaatatgaggttactggtggatcattcgatagtggagggatttgcacaaaacggaagaacggtggtgacatcgagagtgtatccaacgaaggcaatttacaacgctgcgaaggtgtttttgttcaacaacgcgaccgggattagggtgaaggcgtcggtcaagatttggaagatggcggaagcagaactcaaccctttcccagttactgggtggacttcttgagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagc

SEQ ID NO: 15 - DNA, 6667 bp, Expression vector pRM03
  misc_feature (3914)..(3914): n = A, C, G, or T
ggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcaccatggcaacccctgaacaacccattacagaccttgaacacgaacccaaccacaaccgcacacccctattggaccacaacgaatcacaacccgtaaagaaacatttgttcttcaaagttctgtctggtgttaccttcatttcattgttctttatttctgcttttttattcattgttttgaaccaacaaaattctaccaatatatcggttaagtactcgcaatccgatcgccttacgtgggaacgaaccgcttttcattttcaaccggccaagaattttatttatgatcccaatggtcaaatgtactacatgggctggtaccatctattctatcaatacaatccatacgcaccggtttggggtaatatgtcatggggtcactccgtatccaaagacatgatcaactggtacgagctacccgtcgctatagtcccgactgaatggtatgatattgagggcgtcttatctgggtccatcacagtgcttcccaacgggcagatctttgcattgtacacggggaatgctaatgacttttcccaattgcaatgcaaagctgtacccgtgaactcatctgacccacttcttgttgagtgggtcaagtacgaagataacccaatcctgtacactccaccagggattgggttaaaagactatagggacccgtcaacagtctggacgggtcctgatggaaagcataggatgatcatgggaactaaacgtggcaatacaggaatgatacttgtttaccataccactgattacacgaactatgagatgttgaatgagcctatgcactcggttcccaataccgatatgtgggaatgcgttgacttttacccggtttcattaaccaacgatagtgcacttgatattgcggcctacgggtcgggtatcaaacacgtgattaaagaaagttgggagggatatgggatggatttctattcaatcgggacttatgacgcatttaacgataaatggactcccgataacccagagttagatgttggtatcgggttgcggtgtgattacggtaggttttttgcatcaaagagtatttttgacccagtgaagaaaaggaggatcacttgggcttatgttggagaatcagataatgctgatgatgacctctccagaggatgggctactatttataatgttggaagaactattgtactagatagaaagaccgggacccatttacttcattggcctgtcgaggaaatcgagagtttgagatacaatggtcaggaatttaaagagatcaaactagagcccggttcaattgctccactcgacataggcaccgctacacagttggacatagttgcaacatttaaggtggatgaggctgcattgaacgcgacaagtgaaaccgatgataacttcgcttgcaccacgagctcaggtgcagttgaaaggggaagtttgggaccatttggtcttgcggttctagctgatggaaccctttccgagttaactccggtttatttctacattgctaaaaaggccgatggaggtgtgtcaacacatttttgtaccgataagctaaggtcatccttggattttgataaggagagagtggtgtacggtagcactgttcctgtgttagatgatgaagaactcacaatgaggctattggtggatcattcggtagtcgaggcgtttgcacaaggaggaaggattgccataacatcaagggtgtatccgacgaaagcaatatacgaaggagcgaagttgttcttattcaacaatgccacggatacgagtgtgaaggcatctctcaagatttggcaaatggcttctgcccaaattcatcaatacgagtttaattaggc

SEQ ID NO: 16 - DNA, 34 bp, Artificial
  linker (1)..(34): linker
ggcgcgccaagcttggatccgtcgacggcgcgcc

SEQ ID NO: 17 - DNA, 8890 bp, Expression vector pJMS01
ggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcaccatggcaacccctgaacaacccattacagaccttgaacacgaacccaaccacaaccgcacacccctattggaccacaacgaatcacaacccgtaaagaaacatttgttcttcaaagttctgtctggtgttaccttcatttcattgttctttatttctgcttttttattcattgttttgaaccaacaaaattctaccaatatatcggttaagtactcgcaatccgatcgccttacgtgggaacgaaccgcttttcattttcaaccggccaagaattttatttatgatcccaatggtcaaatgtactacatgggctggtaccatctattctatcaatacaatccatacgcaccggtttggggtaatatgtcatggggtcactccgtatccaaagacatgatcaactggtacgagctacccgtcgctatagtcccgactgaatggtatgatattgagggcgtcttatctgggtccatcacagtgcttcccaacgggcagatctttgcattgtacacggggaatgctaatgacttttcccaattgcaatgcaaagctgtacccgtgaactcatctgacccacttcttgttgagtgggtcaagtacgaagataacccaatcctgtacactccaccagggattgggttaaaagactatagggacccgtcaacagtctggacgggtcctgatggaaagcataggatgatcatgggaactaaacgtggcaatacaggaatgatacttgtttaccataccactgattacacgaactatgagatgttgaatgagcctatgcactcggttcccaataccgatatgtgggaatgcgttgacttttacccggtttcattaaccaacgatagtgcacttgatattgcggcctacgggtcgggtatcaaacacgtgattaaagaaagttgggagggatatgggatggatttctattcaatcgggacttatgacgcatttaacgataaatggactcccgataacccagagttagatgttggtatcgggttgcggtgtgattacggtaggttttttgcatcaaagagtatttttgacccagtgaagaaaaggaggatcacttgggcttatgttggagaatcagataatgctgatgatgacctctccagaggatgggctactatttataatgttggaagaactattgtactagatagaaagaccgggacccatttacttcattggcctgtcgaggaaatcgagagtttgagatacaatggtcaggaatttaaagagatcaaactagagcccggttcaattgctccactcgacataggcaccgctacacagttggacatagttgcaacatttaaggtggatgaggctgcattgaacgcgacaagtgaaaccgatgataacttcgcttgcaccacgagctcaggtgcagttgaaaggggaagtttgggaccatttggtcttgcggttctagctgatggaaccctttccgagttaactccggtttatttctacattgctaaaaaggccgatggaggtgtgtcaacacatttttgtaccgataagctaaggtcatccttggattttgataaggagagagtggtgtacggtagcactgttcctgtgttagatgatgaagaactcacaatgaggctattggtggatcattcggtagtcgaggcgtttgcacaaggaggaaggattgccataacatcaagggtgtatccgacgaaagcaatatacgaaggagcgaagttgttcttattcaacaatgccacggatacgagtgtgaaggcatctctcaagatttggcaaatggcttctgcccaaattcatcaatacgagtttaattaggc

SEQ ID NO: 18 - DNA, 8965 bp, Expression vector pRM02
ggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgccatggcttcatctaccaccacctcccctctcattctccacgatgatcctgaaaacctccaggaacccaccggatttacgggggttcgtcgtccatccatcgcaaaagcgctttgcgtaacccttgtttcggttatggtaatctgtggtctggttgctgtaatcagcaaccagacacaggtaccacaagtagccaacagccatcaaggtgccgccaccacattcacaactcagttgccaaaaatagatatgaaacgggttccgggagagttggattcgggtgctgatgtccaatggcaacgctccgcttatcattttcaacctgacaaaaactacattagtgatcctgatggcccaatgtatcacatgggatggtaccatctattttatcagtacaacccagaatctgccatatggggcaacatcacatggggtcactccgtatccaaagacatgatcaactggttccatctccctttcgccatggttccggaccattggtacgacatcgaaggcgtcatgacaggttccgccacagtcctcccaaacggtgagatcatcatgctttacacgggcaatgcgtacgatctctcccaagtacaatgcttagcgtacgcagtcaactcatcagatccacttcttatagagtggaaaaaatacgaaggcaacccggttttattgccgccgccaggggtgggttacaaggattttcgggacccatctacattgtggctgggccccgatggtgaatatagaatggtaatggggtccaagcacaacgagactattggttgtgctttgatttaccataccactaattttacgcattttgaattgaatgaggaggtgcttcatgcggtcccacatactggtatgtgggaatgcgttgatctttatccggtatccaccacacacacaaacgggttggacatggtggataatgggccaaatgtaaaatacgtgttgaaacaaagtggggatgaagatcgccatgattggtatgcgattggaagttatgattgggtgaatgataagtggtacccggatgacccggaaaacgatgtgggtatcgggttaagatacgattacggaaagttttatgcgtccaagacgttttatgaccaacataagaaaaggagggtcctttggggctatgttggagaaaccgatcccgaaaagtatgaccttacaaagggatgggctaacatattgaatattccaaggaccgtcgttttggacacgaaaactaaaaccaatttgattcaatggccaattgaggaaaccgaaaaacttaggtcgaaaaagtatgataaatttgtagatgtggagcttcgacccgggtcactcattcccctcgagataggtacagccacacagttggatatagttgcgacattcgaagttgatcaaatgatgttggaatcaacgctagaagccgatgttctattcaactgcacgactagtgttggctcagttggaaggggcgtgttgggaccgtttggtgtggtggttctagctgatgcccagcgcaccgaacaacttcctgtgtatttctatattgcaaaagataccgacgggacgtcaagaacctacttttgtgctgatgaaacaagatcatccaaggatgtagacgtggggaaatgggtgtatggaagcagtgttcctgtcctccctaacgaaaagtacaatatgaggttactggtggatcattcgatagtggagggatttgcacaaaacggaagaacggtggtgacatcgagagtgtatccaacgaaggcaatttacaacgctgcgaaggtgtttttgttcaacaacgcgaccgggattagggtgaaggcgtcggtcaagatttggaagatggcggaagcagaactcaaccctttcccagttactgggtggacttcttgagc

SEQ ID NO: 19 - DNA, 13092 bp, Expression vector pRM01
  misc_feature (10061)..(10061): n = A, C, G, or T
tcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcaccatggcaacccctgaacaacccattacagaccttgaacacgaacccaaccacaaccgcacacccctattggaccacaacgaatcacaacccgtaaagaaacatttgttcttcaaagttctgtctggtgttaccttcatttcattgttctttatttctgcttttttattcattgttttgaaccaacaaaattctaccaatatatcggttaagtactcgcaatccgatcgccttacgtgggaacgaaccgcttttcattttcaaccggccaagaattttatttatgatcccaatggtcaaatgtactacatgggctggtaccatctattctatcaatacaatccatacgcaccggtttggggtaatatgtcatggggtcactccgtatccaaagacatgatcaactggtacgagctacccgtcgctatagtcccgactgaatggtatgatattgagggcgtcttatctgggtccatcacagtgcttcccaacgggcagatctttgcattgtacacggggaatgctaatgacttttcccaattgcaatgcaaagctgtacccgtgaactcatctgacccacttcttgttgagtgggtcaagtacgaagataacccaatcctgtacactccaccagggattgggttaaaagactatagggacccgtcaacagtctggacgggtcctgatggaaagcataggatgatcatgggaactaaacgtggcaatacaggaatgatacttgtttaccataccactgattacacgaactatgagatgttgaatgagcctatgcactcggttcccaataccgatatgtgggaatgcgttgacttttacccggtttcattaaccaacgatagtgcacttgatattgcggcctacgggtcgggtatcaaacacgtgattaaagaaagttgggagggatatgggatggatttctattcaatcgggacttatgacgcatttaacgataaatggactcccgataacccagagttagatgttggtatcgggttgcggtgtgattacggtaggttttttgcatcaaagagtatttttgacccagtgaagaaaaggaggatcacttgggcttatgttggagaatcagataatgctgatgatgacctctccagaggatgggctactatttataatgttggaagaactattgtactagatagaaagaccgggacccatttacttcattggcctgtcgaggaaatcgagagtttgagatacaatggtcaggaatttaaagagatcaaactagagcccggttcaattgctccactcgacataggcaccgctacacagttggacatagttgcaacatttaaggtggatgaggctgcattgaacgcgacaagtgaaaccgatgataacttcgcttgcaccacgagctcaggtgcagttgaaaggggaagtttgggaccatttggtcttgcggttctagctgatggaaccctttccgagttaactccggtttatttctacattgctaaaaaggccgatggaggtgtgtcaacacatttttgtaccgataagctaaggtcatccttggattttgataaggagagagtggtgtacggtagcactgttcctgtgttagatgatgaagaactcacaatgaggctattggtggatcattcggtagtcgaggcgtttgcacaaggaggaaggattgccataacatcaagggtgtatccgacgaaagcaatatacgaaggagcgaagttgttcttattcaacaatgccacggatacgagtgtgaaggcatctctcaagatttggcaaatggcttctgcccaaattcatcaatacgagtttaattaggcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcgccatggcttcatctaccaccacctcccctctcattctccacgatgatcctgaaaacctccaggaacccaccggatttacgggggttcgtcgtccatccatcgcaaaagcgctttgcgtaacccttgtttcggttatggtaatctgtggtctggttgctgtaatcagcaaccagacacaggtaccacaagtagccaacagccatcaaggtgccgccaccacattcacaactcagttgccaaaaatagatatgaaacgggttccgggagagttggattcgggtgctgatgtccaatggcaacgctccgcttatcattttcaacctgacaaaaactacattagtgatcctgatggcccaatgtatcacatgggatggtaccatctattttatcagtacaacccagaatctgccatatggggcaacatcacatggggtcactccgtatccaaagacatgatcaactggttccatctccctttcgccatggttccggaccattggtacgacatcgaaggcgtcatgacaggttccgccacagtcctcccaaacggtgagatcatcatgctttacacgggcaatgcgtacgatctctcccaagtacaatgcttagcgtacgcagtcaactcatcagatccacttcttatagagtggaaaaaatacgaaggcaacccggttttattgccgccgccaggggtgggttacaaggattttcgggacccatctacattgtggctgggccccgatggtgaatatagaatggtaatggggtccaagcacaacgagactattggttgtgctttgatttaccataccactaattttacgcattttgaattgaatgaggaggtgcttcatgcggtcccacatactggtatgtgggaatgcgttgatctttatccggtatccaccacacacacaaacgggttggacatggtggataatgggccaaatgtaaaatacgtgttgaaacaaagtggggatgaagatcgccatgattggtatgcgattggaagttatgattgggtgaatgataagtggtacccggatgacccggaaaacgatgtgggtatcgggttaagatacgattacggaaagttttatgcgtccaagacgttttatgaccaacataagaaaaggagggtcctttggggctatgttggagaaaccgatcccgaaaagtatgaccttacaaagggatgggctaacatattgaatattccaaggaccgtcgttttggacacgaaaactaaaaccaatttgattcaatggccaattgaggaaaccgaaaaacttaggtcgaaaaagtatgataaatttgtagatgtggagcttcgacccgggtcactcattcccctcgagataggtacagccacacagttggatatagttgcgacattcgaagttgatcaaatgatgttggaatcaacgctagaagccgatgttctattcaactgcacgactagtgttggctcagttggaaggggcgtgttgggaccgtttggtgtggtggttctagctgatgcccagcgcaccgaacaacttcctgtgtatttctatattgcaaaagataccgacgggacgtcaagaacctacttttgtgctgatgaaacaagatcatccaaggatgtagacgtggggaaatgggtgtatggaagcagtgttcctgtcctccctaacgaaaagtacaatatgaggttactggtggatcattcgatagtggagggatttgcacaaaacggaagaacggtggtgacatcgagagtgtatccaacgaaggcaatttacaacgctgcgaaggtgtttttgttcaacaacgcgaccgggattagggtgaaggcgtcggtcaagatttggaagatggcggaagcagaactcaaccctttcccagttactgggtggacttcttgagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccg

SEQ ID NO: 20 - DNA, 13092 bp, Expression vector pRM04
  misc_feature (10136)..(10136): n = A, C, G, or T
tcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgccatggcttcatctaccaccacctcccctctcattctccacgatgatcctgaaaacctccaggaacccaccggatttacgggggttcgtcgtccatccatcgcaaaagcgctttgcgtaacccttgtttcggttatggtaatctgtggtctggttgctgtaatcagcaaccagacacaggtaccacaagtagccaacagccatcaaggtgccgccaccacattcacaactcagttgccaaaaatagatatgaaacgggttccgggagagttggattcgggtgctgatgtccaatggcaacgctccgcttatcattttcaacctgacaaaaactacattagtgatcctgatggcccaatgtatcacatgggatggtaccatctattttatcagtacaacccagaatctgccatatggggcaacatcacatggggtcactccgtatccaaagacatgatcaactggttccatctccctttcgccatggttccggaccattggtacgacatcgaaggcgtcatgacaggttccgccacagtcctcccaaacggtgagatcatcatgctttacacgggcaatgcgtacgatctctcccaagtacaatgcttagcgtacgcagtcaactcatcagatccacttcttatagagtggaaaaaatacgaaggcaacccggttttattgccgccgccaggggtgggttacaaggattttcgggacccatctacattgtggctgggccccgatggtgaatatagaatggtaatggggtccaagcacaacgagactattggttgtgctttgatttaccataccactaattttacgcattttgaattgaatgaggaggtgcttcatgcggtcccacatactggtatgtgggaatgcgttgatctttatccggtatccaccacacacacaaacgggttggacatggtggataatgggccaaatgtaaaatacgtgttgaaacaaagtggggatgaagatcgccatgattggtatgcgattggaagttatgattgggtgaatgataagtggtacccggatgacccggaaaacgatgtgggtatcgggttaagatacgattacggaaagttttatgcgtccaagacgttttatgaccaacataagaaaaggagggtcctttggggctatgttggagaaaccgatcccgaaaagtatgaccttacaaagggatgggctaacatattgaatattccaaggaccgtcgttttggacacgaaaactaaaaccaatttgattcaatggccaattgaggaaaccgaaaaacttaggtcgaaaaagtatgataaatttgtagatgtggagcttcgacccgggtcactcattcccctcgagataggtacagccacacagttggatatagttgcgacattcgaagttgatcaaatgatgttggaatcaacgctagaagccgatgttctattcaactgcacgactagtgttggctcagttggaaggggcgtgttgggaccgtttggtgtggtggttctagctgatgcccagcgcaccgaacaacttcctgtgtatttctatattgcaaaagataccgacgggacgtcaagaacctacttttgtgctgatgaaacaagatcatccaaggatgtagacgtggggaaatgggtgtatggaagcagtgttcctgtcctccctaacgaaaagtacaatatgaggttactggtggatcattcgatagtggagggatttgcacaaaacggaagaacggtggtgacatcgagagtgtatccaacgaaggcaatttacaacgctgcgaaggtgtttttgttcaacaacgcgaccgggattagggtgaaggcgtcggtcaagatttggaagatggcggaagcagaactcaaccctttcccagttactgggtggacttcttgagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcaccatggcaacccctgaacaacccattacagaccttgaacacgaacccaaccacaaccgcacacccctattggaccacaacgaatcacaacccgtaaagaaacatttgttcttcaaagttctgtctggtgttaccttcatttcattgttctttatttctgcttttttattcattgttttgaaccaacaaaattctaccaatatatcggttaagtactcgcaatccgatcgccttacgtgggaacgaaccgcttttcattttcaaccggccaagaattttatttatgatcccaatggtcaaatgtactacatgggctggtaccatctattctatcaatacaatccatacgcaccggtttggggtaatatgtcatggggtcactccgtatccaaagacatgatcaactggtacgagctacccgtcgctatagtcccgactgaatggtatgatattgagggcgtcttatctgggtccatcacagtgcttcccaacgggcagatctttgcattgtacacggggaatgctaatgacttttcccaattgcaatgcaaagctgtacccgtgaactcatctgacccacttcttgttgagtgggtcaagtacgaagataacccaatcctgtacactccaccagggattgggttaaaagactatagggacccgtcaacagtctggacgggtcctgatggaaagcataggatgatcatgggaactaaacgtggcaatacaggaatgatacttgtttaccataccactgattacacgaactatgagatgttgaatgagcctatgcactcggttcccaataccgatatgtgggaatgcgttgacttttacccggtttcattaaccaacgatagtgcacttgatattgcggcctacgggtcgggtatcaaacacgtgattaaagaaagttgggagggatatgggatggatttctattcaatcgggacttatgacgcatttaacgataaatggactcccgataacccagagttagatgttggtatcgggttgcggtgtgattacggtaggttttttgcatcaaagagtatttttgacccagtgaagaaaaggaggatcacttgggcttatgttggagaatcagataatgctgatgatgacctctccagaggatgggctactatttataatgttggaagaactattgtactagatagaaagaccgggacccatttacttcattggcctgtcgaggaaatcgagagtttgagatacaatggtcaggaatttaaagagatcaaactagagcccggttcaattgctccactcgacataggcaccgctacacagttggacatagttgcaacatttaaggtggatgaggctgcattgaacgcgacaagtgaaaccgatgataacttcgcttgcaccacgagctcaggtgcagttgaaaggggaagtttgggaccatttggtcttgcggttctagctgatggaaccctttccgagttaactccggtttatttctacattgctaaaaaggccgatggaggtgtgtcaacacatttttgtaccgataagctaaggtcatccttggattttgataaggagagagtggtgtacggtagcactgttcctgtgttagatgatgaagaactcacaatgaggctattggtggatcattcggtagtcgaggcgtttgcacaaggaggaaggattgccataacatcaagggtgtatccgacgaaagcaatatacgaaggagcgaagttgttcttattcaacaatgccacggatacgagtgtgaaggcatctctcaagatttggcaaatggcttctgcccaaattcatcaatacgagtttaattaggcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccg

(19) United States
(12) Patent Application Publication
(10) Pub. No.: US 2004/0073975 A1
(43) Pub. Date: Apr. 15, 2004

(54) Product of novel fructose polymers in embryos of transgenic plants

(76) Inventor: Johan M. Stoop, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/644,335

(22) Filed: Aug. 20, 2003

Related U.S. Application Data
(60) Provisional application No. 60/404,844, filed on Aug. 21, 2002.

(51) Int. Cl. A01H 1/00; A01H 5/00; C12N 9/10; C08B 37/00

(52) U.S. Cl. 800/284; 536/123; 435/193; 800/320.1; 800/312

(57) ABSTRACT

This invention includes a recombinant DNA molecule comprising a sequence for a fructosyltransferase capable of producing a novel fructan, a method for producing transgenic plants exhibiting a novel fructan, transformed plants and plant parts comprising said novel fructan, and products prepared therefrom.

This application claims the benefit of U.S. Provisional Application No. 60/404,844 filed Aug. 21, 2002. The entire content of the provisional application is herein incorporated by reference for all purposes.

### FIELD OF INVENTION

This invention relates to the field of plant molecular biology. The present invention includes methods for producing transgenic plant species showing a novel fructan profile, transformed plants or plant parts showing said novel fructan profile, and products prepared therefrom.

### BACKGROUND OF INVENTION

The major reserve carbohydrates found in vascular plants are sucrose, starch, cellulose and fructan. Sucrose is most commonly purified from sucrose-producing plants and used as a sweetener. Starch and cellulose are currently used in numerous food and non-food applications in their native form, but their usefulness is greatly expanded by enzymatic or chemical modification. Fructan has commercial applications in the industrial, medical, food and feed industries.

Fructan includes linear or branched polymers of repeating fructose residues connected by β2-1 and/or β2-6 fructosyl-fructose linkages, optionally including one terminal glucosyl unit. The number of residues contained in an individual fructan polymer is also known as the degree of polymerization, or DP, and varies greatly depending on the source from which the fructan is isolated. For example, fructan isolated from fungal species, such as Aspergillus syndowi, may contain two or three fructose residues, fructan obtained from plants have low to intermediate DP (3 to 200), and fructan found in bacteria, such as Bacillus amyloliquefaciens or Streptococcus mutans, have a DP of 5,000 or greater.

Fructan accumulation in plants is highly sensitive to environmental changes. Exposure to drought or frost dramatically alters the quality of the fructan accumulated (Praznik and Beck (1987) Agr. Biol. Chem. 51:1593-1599). Traditional breeding programs could, in theory, result in varieties with reduced quality losses due to environmental changes. However, programs of this type are very time consuming, are not in place at this time, and would likely be implemented only when the fructan industry proves them to be viable.

The ability to produce fructan of the desired size in large amounts in crops of agronomic importance, such as corn and/or soybean, will reduce fructan production costs. Fructan production in corn for example, allows the utilization of the corn byproducts (oil, meal and gluten feed) in addition to removing the costs of converting glucose to fructose. Hydrolysis of fructan into individual fructose residues results in a product consisting of at least 99% fructose. This highly pure product provides an alternative to the inefficient isomerization step, usually used to convert glucose isolated from starch to fructose, and eliminates the need for fructose enrichment by ion exchange chromatography. Crystallization of fructose is simplified by starting with material that consists of 99%(+) fructose. Availability of fructose at a competitive cost would allow it, easily dehydrated to 5-hydroxymethyl-furfural (HMF), to be utilized as a building block for pharmaceuticals, such as ranitidine and Zantac®. HMF may also be used as starting material for polymers such as Kevlar®, and Nomex®, in addition to the potential for use in opto-electronic devices, due to the special optical effects of the furan nucleus (Schiweck et al. (1992) in Carbohydrates as Organic Raw Materials, Lichtenthaler ed., VCH Press, NY, pp. 72-82). HMF may be converted into carbocyclic and heterocyclic compounds, creating a role in almost every part of applied chemistry, if only its purity could be combined with increased production and reduced cost.

The fructan produced in plants differ structurally depending on the linkages of the fructosyl residues. Linear β2-1 linkages of fructose residues form inulin(s) found in chicory, sunflower, and Jerusalem artichoke, among others. Linear β2-6 linkages of fructose residues form levan(s) found in some grasses. Mixed levans, also called graminans, have a mixture of β2-1 and β2-6 linkages and are found for example in wheat and barley. Fructose residues connected by β2-1 and β2-6 linkages on carbons 1 and 6 of the glucose moiety of the sucrose molecule form the inulin neoseries. These fructan are found in onion, leek, and asparagus, for example. The levan neoseries contain predominantly β2-6 linkages of fructosyl residues on either end of the glucosyl moiety of the sucrose molecule and are found in oat, for example.

Several models have been proposed for the formation of the different plant fructan. In one of these models (Vijn, I. and Smeekens, S. (1999) Plant Phys. 120:351-359) conversion of sucrose to 1-kestose (also called isoketose) is catalyzed by sucrose:sucrose 1-fructosyltransferase (1-SST; EC 2.4.1.99) and conversion to 6-kestose is catalyzed by sucrose:fructan 6-fructosyltransferase (6-SFT; EC 2.4.1.10). Elongation of 6-kestose to levans is also catalyzed by 6-SFT. Addition of a fructosyl unit from sucrose to 1-kestose produces neokestose when catalyzed by fructan:fructan 6G-fructosyltransferase (6G-FFT) and produces bifurcose when catalyzed by 6-SFT. Conversion of neokestose to the levan neoseries and of bifurcose to mixed-type levans is also catalyzed by 6-SFT. This last conversion has been suggested to be by the action of exohydrolase or fructan:fructan 1-fructosyltransferase (1-FFT, EC 2.4.1.100). 1-FFT catalyzes the production of inulin, inulin neoseries, and mixed type levans.

In contrast to fructan-producing plants, which use at least two fructosyltransferases (FTFs) to produce fructan of low to intermediate DP (DP3 to 200), bacteria produce high-DP fructan (DP>5000) using a single FTF. Differences in the specificity for donor and acceptor molecules have also been reported for bacterial and plant FTFs. The bacterial enzymes are known to have a hydrolase activity by which water is used as a fructosyl acceptor resulting in the release of significant amounts of fructose from sucrose. This hydrolase activity is referred to as invertase activity. Hydrolytic activities have been suggested for some SSTs (Koops and Jonker (1996) Plant Physiol. 110:1167-1175) but have yet to be found in FFTs (Chambert, R. and Petit-Glatron, M. (1993) in Inulin and Inulin Containing Crops, A. Fuchs ed. Elsevier Press, Amsterdam. pp. 259-266).

Fructose, liberated from sucrose by invertase activity, cannot be used to increase the length of a fructan polymer. Bacterial FTFs, therefore, convert sucrose to fructan less efficiently than do the plant enzymes. Plant and bacterial FTFs also differ in their affinity for sucrose, the sole substrate. Jerusalem artichoke SST has a Km for sucrose reported to be approximately 100 mM (Koops, A. and Jonker, H., (1994) J. Exp. Bot. 45:1623-1631). By contrast, the bacterial enzyme has a much lower Km of approximately 20 mM (Chambert, R. and Petit-Glatron, M. (1991) Biochem. J. 279:35-41). This difference may have a critical effect on fructan synthesis, resulting in higher or lower levels of accumulation, depending on the concentration of sucrose in the cell.

In an attempt to produce fructan in crops of agronomic importance, transgenic plants expressing bacterial FTFs have been produced. Oligosaccharides and fructan are produced in differing amounts when the FTF is expressed under control of a constitutive promoter, an endosperm specific promoter, or a tuber specific promoter and is directed to different subcellular locations. Direction to the vacuole, the chloroplast, and the endosperm proved to be the most efficient in producing levan in transgenic plants. In PCT publication No. WO 89/12386, published Dec. 28, 1989, FTF activity was found in transgenic tomato plants prepared expressing B subtilis levansucrase under the control of the mas promoter and having an apoplast-signal sequence, or expressing the L. mesenteroides dextransucrase under the control of the constitutive cauliflower mosaic virus 35S (CaMV 35S) promoter. While potato and tobacco plants expressing E. amylovora levansucrase under the control of the CaMV 35S promoter did not produce detectable fructan when the enzyme was expressed in the cytoplasm, direction of the enzyme to the apoplasm or the tubers produced detectable levans (PCT publication No. WO 94/04692, published Mar. 3, 1994). PCT publication No. WO 94/14970, published Jul. 7, 1994, shows that expression in tobacco or potato of B. subtilis SacB or S. mutans ftf under the control of the CaMV 35S promoter resulted in the production of bacterial-like fructan regardless of the subcellular location of the enzyme (vacuole, apoplast, or cytoplasm). Production of fructan by means of the S. mutans ftf gene in tobacco resulted in only very low amounts of DP3. Smeekens, J. C. et al. (PCT publication No. WO 96/01904 published Jan. 25, 1996).

The results using B. amyloliquefaciens SacB in transgenic tobacco, potato, or corn plants are not consistent as set forth in PCT publication No. WO 95/13389, published May 18, 1995. In tobacco, expression of the bacterial FTF under the control of the SSU promoter and directed to the cytoplasm proved to be detrimental to the plants, expression under the control of the inducible 2-1.3 promoter produced small amounts of fructan when directed to the chloroplast and produced small amounts of enzyme when directed to the vacuole. In the same publication it was shown that when the bacterial FTF is expressed under the control of the tuber-specific patatin promoter and directed to the cytoplasm no detectable levels of fructan are produced, yet detectable levels of fructan are produced when the enzyme is directed to the vacuole. Furthermore, the same publication shows that expression in corn of the bacterial FTF under the control of the endosperm-specific 10 kD zein promoter produced fructan when directed to the cytoplasm or the vacuole of both, dent maize and starch mutant corn lines.

After inhibition of starch production, due to expression of the antisense ADP-glucose pyrophosphorylase gene, targeted expression of the Erwinia amylovora levansucrase to the apoplasm, vacuole, or cytosol of potato yields varied results as set forth in PCT publication No. WO 94/04692 published Mar. 3, 1994. WO 94/04692 shows fructan accumulating to an appreciable level only in plants where the transgene was targeted to the apoplasm or the vacuole. Expression of the levansucrase in the apoplasm resulted in smaller tubers while its expression in the vacuole did not change the tuber morphology. The fructan produced in the transgenic plants had similar characteristics to the fructan naturally produced by the bacteria. Targeting of SacB gene of B. subtilis to the plastid resulted in fructan accumulation over 10% dry weight in tobacco and up to 5% dry weight in potatoes grown during winter. The fructan produced in these transgenic plants are believed to be associated with the starch granules (PCT publication No. WO 97/29186 published Aug. 14, 1997). Transgenic potato expressing an FTF from S. mutans under the patatin promoter has been reported to produce inulin, useful for industrial applications (PCT publication No. WO 97/42331 published Nov. 13, 1997).

Transformation of plants with DNA sequences encoding plant FTFs have also been reported. Production of fructan using plant derived FTFs in transgenic dicots has been successful to a limited extent in tobacco, petunia and potato. Leaves of transgenic petunia plants, expressing Jerusalem artichoke 1-SST, and leaves of transgenic potato plants expressing the Jerusalem artichoke 1-SST and 1-FFT, produced only small amounts of tri- tetra-, and penta-saccharides. The tri- and tetra-saccharides are detectable by thin layer chromatography while the penta-saccharides are detectable only by HPAEC analyses (PCT publication No. WO 96/21023 published Jul. 11, 1996). Transgenic petunia plants expressing Jerusalem artichoke 1-SST and 1-FFT produced fructan molecules of DP up to 25 (van der Meer, I. M., et al. (1998) Plant J. 15:489-500). Smeekens, J. C. et al. (PCT publication No. WO 96/01904 published Jan. 25, 1996) suggest preparing transgenic plants with sequences encoding onion SST, barley 6-SFT, Jerusalem artichoke FFT, potato FFT, or mutants of these genes to produce fructan for use as sweeteners. Sequences encoding onion SST, barley 6-SFT, and potato FFT were identified, the cDNAs encoding the FTFs were isolated and used for the preparation of transformation vectors which were then used to create transgenic plants. No data was reported with regard to the amount of the fructan made.

Transgenic potato expressing the artichoke (Cynare scolymus) SST under the direction of the patatin B33 promoter or the CaMV 35S promoter resulted in transgenic plants producing limited amounts of the DP-3,1-kestose (PCT publication No. WO 98/39460 published Sep. 11, 1998). Transgenic potato plants expressing globe artichoke (Cynara scolymus) 1-SST and 1-FFT produced inulin molecules of DP>60, similar to the inulin profile found in the globe artichoke. Total fructan accumulation in the tubers averaged around 35 μmol/g fresh weight artichoke (Hellwege, E. M., et al. (2000) Proc. Natl. Acad. Sci. U.S.A. 97:8699-8704; PCT publication No. WO 99/24593 published May 20, 1999).

Production of fructan using plant derived FTFs in transgenic monocots has been successfully accomplished in maize. Targeting the fructan synthesizing enzymes 1-SST and 1-FFT of Jerusalem artichoke to the vacuole of maize endosperm (PCT publication No WO 99/46395 published Sep. 16, 1999) resulted in production of low amounts of inulin-type fructan.

As can be seen above efforts to express fructan in plant species in which they are not ordinarily produced at high levels show varying levels of success, as measured by the level of fructan obtained. Aside from the particular fructosyltransferase used, there are three major variables related to the expression system: the plant species, the organ (seed, tuber, leaf, throughout the plant) and intracellular location (vacuole, plastid, cytoplasm, apoplast) in which or to which the fructosyltransferases are expressed or targeted. This variation in observed fructan level presumably relates in large part to the biological and physiological complexities of the different systems used. For example, seeds are a natural storage organ of plants and thus may initially seem a likely place to attempt to accumulate fructan. Seeds contain high levels of proteins, carbohydrates (usually starch), and lipids that are stored for use by the young plant upon germination. It is these storage products that give seeds, when used as grain, their economic value. But seeds are not uniform across species. The relative levels of the different types of storage products varies; see for example Table 19.2, page 1029 of Biochemistry and Molecular Biology of Plants (B. Buchanen, W. Gruissem, and R. Jones, American Society of Plant Physiologists, 2000; hereafter BMBP). Corn has very high levels of carbohydrates, a pattern that approximately holds for the major cereal crops. Soybean, a dicot, has little soluble carbohydrate and is high in protein, with moderate amounts of oil. The soluble carbohydrate pool in soybean seeds is comprised mainly of raffinose family oligsaccharides (39%) and sucrose (54%). Starch accounts for less than 1% of the mature seed dry weight (dry wt), and hexose sugars are barely detectable (Yazdi-Samadi B, et al. (1977), Agr. J. 69:481-486). Potato tubers, another storage organ, store mainly starch. This difference is perhaps reflective of physiological differences. Potato tuber initiation, growth, and development are characterized by significant modifications in hexose and sucrose concentrations and in the ratios of hexose:sucrose and glucose:fructose, but during the main period of reserve product accumulation the hexose levels are in general higher than in soybean. In contrast to both these examples, other dicots such as rapeseed have higher oil than protein levels in their seed. While not intending to be bound by any theory or theories of operation, these differences in storage reserve composition and underlying physiology may be related to differential expression of large numbers of genes at different times in development, as shown by Ruuska, S. A., et al. ((2002) Plant Cell 14:1191-1206) in the model system Arabidopsis. Plants do not compensate the turning off, by mutation or transgenic means, of the pathway for one reserve component by producing higher levels of another component. That fact, and the differences in storage compounds between species, make the introduction of genes encoding enzymes in the pathway for a different storage reserve product not usually found in a species, for example fructan, unpredictable between different species. It is not guaranteed that appropriate metabolic precursors for the desired product will be available.

Seeds have the added complexity that they are genetically non-uniform as they result from a unique double fertilization event. When the pollen tube reaches the ovule, two sperm cells are released. One fertilizes the egg cell, giving rise to the zygote. The second sperm cell fertilizes a unique structure called the central cell, which is diploid. The resulting triploid fertilization product gives rise to the triploid endosperm. (BMBP page 1022). This endosperm undergoes different fates in different plant species. In most dicots the endosperm serves a transient role and is much reduced or even essentially gone in the mature seed. By contrast, in many monocots, in particular the cereal crops, the endosperm is the main storage organ of the seed and, by weight, forms the larger part of the seed. Completely different genes are expressed in the endosperm and embryo in cereals such as corn. For example, while neither seed portion is rich in proteins, the endosperm seed proteins are of the prolaminin class (zeins), while the embryo contains globulins. Promoters of these two classes of proteins thus can be used to direct expression in one part of the seed or the other. In fact protein is a minor component; the endosperm is rich in starch; the embryo is rich in oil, however, the embryo is so small relative to the endosperm that corn grain is overall much richer in starch. One wishing to express a novel storage product in corn seeds thus must decide whether to express the relevant genes in the embryo or endosperm. As the endosperm comprises the larger part of the seed, endosperm specific promoters have often been chosen, but the differences in physiology between the two parts of the seed mean that this is not necessarily always the best choice. The present invention demonstrates that surprisingly high levels, on a seed basis, of fructan is obtained by expression of fructosyltransferases in the embryo instead of the endosperm of corn seeds.

In dicot species, where the endosperm is much reduced or degraded completely, expression of exogenous genes is generally done in the embryo. The embryo develops from the zygote. The developing embryo soon itself develops different tissues and organs (described in BMBP, pp 1024 et seq.). The embryo axis contains the root and shoot meristems that will eventually form the new plant. The cotyledons (called the scutellum in monocots) serve as the storage organs of the embryo, serving the role fulfilled by the endosperm in the cereals. The present invention also demonstrates that fructan is obtained by expression of fructosyltransferases in soybean embryos.

### SUMMARY OF INVENTION

The present invention includes a plant and plant part comprising at least one recombinant DNA molecule comprising an embryo specific promoter operably linked to at least a portion of at least one coding sequence for a plant fructosyltransferase, operably linked to a vacuole targeting sequence, said molecule sufficient to express a protein capable of producing fructan having a degree of polymerization of at least three, in an embryo of the plant, or any progeny thereof, wherein the progeny comprise said molecule.

The present invention also includes a recombinant DNA molecule comprising an embryo specific promoter operably linked to at least a portion of at least one coding sequence for a fructosyltransferase, operably linked to a vacuole targeting sequence, the molecule sufficient to express a protein capable of producing fructan in an embryo cell.

Another embodiment of the present invention is a method of producing fructan in a plant comprising constructing at least one recombinant DNA molecule comprising an embryo specific promoter operably linked to a vacuole targeting sequence operably linked to at least a portion of at least one coding sequence for a fructosyltransferase, transforming a plant with said construct, regenerating the plant to produce seed, harvesting seed from the plant, and extracting fructan from the seed.

Yet another embodiment of the present invention is a method of screening transgenic maize tissue expressing embryo targeted genes comprising preparing Type-II maize callus for transformation, transforming callus, selecting transgenic callus lines, regenerating transgenic somatic embryos, and propagating transgenic somatic embryos for both plant production and early trait analyses.

The present invention also includes a foodstuff comprising fructan obtained from a plant comprising at least one recombinant DNA molecule comprising an embryo specific promoter linked to a vacuole targeting sequence operably linked to at least a portion of at least one coding sequence for a fructosyltransferase, the molecule sufficient to express a protein capable of producing fructan of at least DP3 in a grain of the plant, or any progeny thereof, wherein the progeny comprise the molecule.

Yet another embodiment of the present invention is a foodstuff comprising an inulin obtained from a plant comprising at least one recombinant DNA molecule comprising an embryo specific promoter operably linked to a vacuole targeting sequence operably linked to at least a portion of at least one coding sequence for a fructosyltransferase, the molecule sufficient to express a protein capable of producing fructan of at least DP3 in a grain of the plant, or any progeny thereof, wherein the progeny comprise the molecule.

The present invention also includes an industrial product comprising fructan obtained from a plant comprising at least one recombinant DNA molecule comprising an embryo specific promoter operably linked to a vacuole targeting sequence operably linked to at least a portion of at least one coding sequence for a fructosyltransferase, the molecule sufficient to express a protein capable of producing fructan of at least DP3 in a grain of the plant, or any progeny thereof, wherein the progeny comprise the molecule.

Yet another embodiment is grain from the plant of the present invention. Foodstuffs produced by the grain of the plant of the present invention is another embodiment.

### BRIEF DESCRIPTION OF FIGURES AND SEQUENCE LISTING

The invention can be more fully understood from the following detailed description and the accompanying Figures and Sequence Listing which form part of this application.

FIG. 1 depicts a diagram of the GLOBSST01(f) cassette used to express the Jerusalem artichoke SST in transgenic maize embryos. The cassette contains an embryo-specific globulin promoter, a polynucleotide fragment comprising an entire Jerusalem artichoke SST coding region (including the native secretory and vacuolar targeting signals), and a nos 3′ transcription termination region.

FIG. 2 depicts a diagram of the GLOBFFT10(f) cassette used to express the Jerusalem artichoke FFT in transgenic maize embryos. The cassette contains an embryo-specific globulin promoter, a polynucleotide fragment comprising an entire Jerusalem artichoke FFT coding region (including the native secretory and vacuolar targeting signals), and the nos 3′ transcription termination region.

FIG. 3 shows the carbohydrate profile resulting from HPAE/PAD analysis of maize somatic embryos not containing cassettes expressing embryo-specific Jerusalem artichoke SST or FFT. nC=nanoCoulombs.

FIG. 4 shows the carbohydrate profiles resulting from HPAE/PAD analysis of transgenic maize somatic embryos containing intact copies of the GLOBSST01(f) cassette. S=sucrose, Rf=raffinose, DP3=1-kestose and DP4=1-kestotetraose (inulin-type fructose polymers), DP=degree of polymerization. nC=nanoCoulombs.

FIG. 5 shows the carbohydrate profile resulting from HPAE/PAD analysis of transgenic maize somatic embryos containing intact copies of the GLOBSST01(f) and GLOBFFT01(f) cassettes. S=sucrose, Rf=raffinose, DP3 through DP7=inulin polymers containing 1 or more fructose residues. nC=nanoCoulombs.

FIG. 6 shows the carbohydrate profile resulting from HPAE/PAD analysis of individual maize kernels not containing cassettes expressing embryo-specific Jerusalem artichoke SST or FFT. nC=nanoCoulombs.

FIG. 7 shows the carbohydrate profile resulting from HPAE/PAD analysis of individual kernels from transgenic maize lines containing intact copies of the GLOBSST01(f) cassette. S=sucrose, Rf=raffinose, DP3 through DP7=inulin polymers containing 1 or more fructose residues. nC=nanoCoulombs.

FIG. 8 shows the carbohydrate profile resulting from HPAE/PAD analysis of individual kernels from transgenic maize lines containing intact copies of the GLOBSST01(f) and the GLOBFFT01(f) cassettes. S=sucrose, Rf=raffinose, DP3 through DP10=inulin polymers containing 1 or more fructose residues nC=nanoCoulombs.

FIG. 9 depicts a diagram of vector pJMS02 used to express the guayule SST in transgenic soybean embryos. The vector comprises two expression cassettes. One cassette contains a KTi 3 promoter driving the expression of the entire guayule SST coding region (including the native secretory and vacuolar targeting signals) followed by a KTi 3′ transcription terminator. The other cassette contains the T7 promoter driving the expression of HPT, followed by the E. coli T7 RNA polymerase transcription termination signal.

FIG. 10 depicts a diagram of vector pRM03 used to express the guayule 1-FFT in transgenic soybean embryos. The vector comprises two expression cassettes. One expression cassette contains an embryo-specific KTi 3 promoter directing the expression of an entire guayule 1-FFT (including the native secretory and vacuolar targeting signals) followed by a KTi 3′ transcription termination region. The other cassette contains the T7 RNA polymerase promoter directing the expression of HPT followed by a T7 transcription termination signal.

FIG. 11 depicts a diagram of vector pJMS01 used to express the guayule 1-FFT in transgenic soybean embryos. The vector comprises three expression cassettes. One cassette contains an embryo-specific β-conglycinin promoter directing the expression of an entire guayule 1-FFT coding region (including the native secretory and vacuolar targeting signals) followed by a phaseolin 3′ transcription termination region. Another cassette contains the bacterial T7 RNA polymerase promoter directing the expression of HPT followed by the T7 transcription terminator region. The third cassette contains the CaMV 35S promoter directing the expression of HPT followed by the nos 3′ transcription terminator.

FIG. 12 depicts a diagram of vector pRM02 used to express the guayule SST in transgenic soybean embryos. The vector comprises three expression cassettes. One cassette contains an embryo-specific β-conglycinin promoter driving the expression of an entire guayule SST coding region (including the native secretory and vacuolar targeting signals) followed by a phaseolin 3′ transcription termination region. The two other cassettes contain polynucleotide fragments encoding HPT, one under the control of the bacterial T7 RNA polymerase promoter and transcription terminator regions, and one under the CaMV 35S promoter and nos 3′ transcription terminator.

FIG. 13 depicts a diagram of vector pRM01 used to express guayule 1-SST and 1-FFT in transgenic soybean embryos. The vector comprises four expression cassettes. One cassette contains an embryo-specific KTi 3 promoter driving expression of an entire guayule SST coding region followed by a KTi 3′ transcription termination region. Another cassette contains an embryo specific β-conglycinin promoter driving expression of an entire guayule 1-FFT coding region followed by a phaseolin 3′ transcription termination region. The other two cassettes contain polynucleotide fragments encoding HPT, one under the control of the bacterial T7 RNA polymerase promoter and transcription terminator regions, and one under the CaMV 35S promoter and nos 3′ transcription terminator.

FIG. 14 depicts a diagram of vector pRM04 used to express the guayule 1-SST and 1-FFT in transgenic soybean embryos. The vector comprises four expression cassettes. One cassette contains an embryo specific β-conglycinin promoter directing expression of an entire guayule SST coding region followed by the phaseolin 3′ transcription termination region. Another cassette contains the embryo-specific KTi 3 promoter driving expression of an entire guayule 1-FFT coding region followed by a KTi 3′ transcription termination region. The other two cassettes contain polynucleotide fragments encoding HPT, one under the control of the bacterial T7 RNA polymerase promoter and transcription terminator regions, and one under the CaMV 35S promoter and nos 3′ transcription terminator.

FIG. 15 shows the carbohydrate profile resulting from HPAE/PAD analysis of transgenic soybean somatic embryos transformed with expression vectors pRM02 and pRM03 containing the guayule 1-SST and 1-FFT coding sequences. S=sucrose, Rf=raffinose, St=stachyose. DP3 through DP5=inulin polymers containing 1 or more fructose residues. nC=nanoCoulombs.

FIG. 16 shows the carbohydrate profile resulting from HPAE/PAD analysis of soybean somatic embryos not transformed with cassettes expressing guayule SST or FFT coding sequences. nC=nanoCoulombs.

FIG. 17 shows the carbohydrate profile resulting from HPAE/PAD analysis of dried-down soybean somatic embryos transformed with expression vector pRM01 containing nucleotide sequences encoding guayule 1-SST and 1-FFT. nC=nanoCoulombs.

FIG. 18 shows the carbohydrate profile resulting from HPAE/PAD analysis of individual soybean mature seeds transformed with expression vector pRM01 containing nucleotide sequences encoding guayule 1-SST and 1-FFT. nC=nanoCoulombs.

FIG. 19 shows the carbohydrate profile of soybean seeds not containing nucleotide sequences encoding guayule 1-SST and 1-FFT. nC=nanoCoulombs.

The following sequence descriptions and the Sequence Listing attached hereto comply with the rules governing nucleotide and/or amino acid sequence disclosures in patent applications as set forth in 37 C.F.R. §1.821-1.825.

SEQ ID NO:1 is the polynucleotide sequence of plasmid vector GLOBSST01 comprising the GLOBSST01(f) cassette used to express Jerusalem artichoke SST in transgenic maize embryos. The cassette contains an embryo-specific globulin promoter directing the expression of an entire SST coding region (including the native secretory and vacuolar targeting signals) followed by a nos 3′ transcription termination region.

SEQ ID NO:2 is the polynucleotide sequence of plasmid vector pGLOBFFT01 comprising the GLOBFFT01(f) cassette used to express Jerusalem artichoke FFT in transgenic maize embryos. The cassette contains an embryo-specific globulin promoter directing the expression of an entire FFT coding region (including the native secretory and vacuolar targeting signals) followed by the nos 3′ transcription termination region.

SEQ ID NO:3 is the nucleotide sequence of pDETRIC, a polynucleotide fragment containing the bar gene under the control of the CaMV 35S promoter and OCS 3′-end and used to co-transform maize together with pGLOBFFT01(f) and/or pGLOBSST01(f).

SEQ ID NO:4 is the nucleotide sequence of oligonucleotide primer SST-1 used for detection of the Jerusalem artichoke SST in transformed tissue.

SEQ ID NO:5 is the nucleotide sequence of oligonucleotide primer SST-2 used for detection of the Jerusalem artichoke SST in transformed tissue.

SEQ ID NO:6 is the nucleotide sequence of oligonucleotide primer FFT-1 used for detection of the Jerusalem artichoke FFT in transformed tissue.

SEQ ID NO:7 is the nucleotide sequence of oligonucleotide primer FFT-2 used for detection of the Jerusalem artichoke FFT in transformed tissue.

SEQ ID NO:8 is the nucleotide sequence of the oligonucleotide primer SST-3 used for the PCR amplification of the polynucleotide fragment encoding guayule SST from clone epb3c.pk007.n11.

SEQ ID NO:9 is the nucleotide sequence of the oligonucleotide primer SST-4 used for the PCR amplification of the polynucleotide fragment encoding guayule 1-SST from clone epb3c.pk007.n11.

SEQ ID NO:10 is the nucleotide sequence corresponding to the entire cDNA insert in clone epb3c.pk007.n11 encoding an entire guayule 1-SST including secretory and vacuolar signals.

SEQ ID NO:11 is the nucleotide sequence of the oligonulcleotide primer FFT-3 used for the PCR amplification of the polynucleotide fragment encoding Guayle 1-FFT from clone epb3c.pk007.j9.

SEQ ID NO:12 is the nucleotide sequence of the oligonucleotide primer FFT-4 used for the PCR amplification of the polynucleotide fragment encoding guayule 1-FFT from clone epb3c.pk007.j9.

SEQ ID NO:13 is the nucleotide sequence corresponding to the entire cDNA insert in clone epblc.pk007.j9 encoding an entire guayule FFT including secretory and vacuolar signals.

SEQ ID NO:14 is the nucleotide sequence of vector pJMS02 comprising a cassette expressing the guayule SST under control of the embryo-specific KTi 3 promoter and transcription termination regions and a cassette comprising a fragment encoding HPT under control of the E. coli T7 promoter and terminator region.

SEQ ID NO:15 is the nucleotide sequence of vector pRM03 comprising the embryo-specific KTi 3 promoter directing the expression of an entire guayule FFT (including the native secretory and vacuolar targeting signals) followed by a KTi 3′ transcription terminator and the E. coli T7 RNA polymerase promoter directing the expression of HPT followed by a T7 transcription terminator.

SEQ ID NO:16 is the nucleotide sequence of the linker fragment used to introduce sites into the modified plasmid pKS17. In a 5′ to 3′ orientation, this linker fragment contains restriction sites for Asc I, Hind III, Bam HI, Sal I, Asc I.

SEQ ID NO:17 is the nucleotide sequence of vector pJMS01 comprising three expression cassettes. One cassette contains the embryo-specific β-conglycinin promoter operably linked to a polynucleotide fragment encoding an entire guayule FFT coding region (including the native secretory and vacuolar targeting signals) followed by a phaseolin 3′ transcription terminator. Another cassette contains the E. coli T7 RNA polymerase promoter operably linked to a polynucleotide encoding HPT, which is operably linked to the E. coli T7 transcription terminator. A third cassette contains the CaMV 35S promoter operably linked to a polynucleotide encoding HPT, which is operably linked to the nos 3′ transcription terminator.

SEQ ID NO:18 is the nucleotide sequence of vector pRM02 comprising three expression cassettes. One cassette contains the embryo-specific β-conglycinin promoter operably linked to the polynucleotide encoding an entire guayule SST coding region (including the native secretory and vacuolar targeting signals) followed by a phaseolin 3′ transcription terminator. Another cassette contains the E. coli T7 RNA polymerase promoter operably linked to a polynucleotide encoding HPT, followed by the E. coli T7 transcription terminator. A third cassette contains the CaMV 35S promoter operably linked to a polynucleotide encoding HPT, which is operably linked to the nos 3′ transcription terminator.

SEQ ID NO:19 is the nucleotide sequence of vector pRM01 comprising four expression cassettes. One cassette expresses guayule SST under control of the embryo-specific KTi 3 promoter and transcription terminator. Another cassette expresses guayule FFT under control of the embryo-specific β-conglycinin promoter and phaseolin transcription terminator. The other two cassettes express HPT, one under control of the bacterial T7 RNA promoter and one under control of the CaMV 35S promoter.

SEQ ID NO:20 is the nucleotide sequence of vector pRM04 comprising four expression cassettes. One cassette expresses guayule FFT under control of the embryo-specific KTi 3 promoter and transcription terminator. Another cassette expresses guayule SST under control of the embryo-specific β-conglycinin promoter and phaseolin transcription terminator. The other two cassettes express HPT, one under control of the bacterial T7 RNA promoter and one under control of the CaMV 35S promoter.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE PREFERRED EMBODIMENTS OF THE INVENTION

Definitions

In the context of this disclosure, a number of terms should be utilized.

The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof.

The term “isolated” refers to materials, such as nucleic acid molecules and/or proteins, substantially free or otherwise removed from components that normally accompany or interact with the materials in a naturally occurring environment. Isolated polynucleotides may be purified from other nucleic acid sequences, such as and not limited to chromosomal and extrachromosomal DNA and RNA, in a host cell in which they naturally occur, for example. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques. A “recombinant DNA molecule or construct” comprises an isolated polynucleotide operably linked to at least one regulatory sequence. The term also embraces an isolated polynucleotide comprising a region encoding all or part of a functional RNA and at least one of the naturally occurring regulatory sequences directing expression in the source (e.g., organism) from which the polynucleotide was isolated, such as, but not limited to, an isolated polynucleotide comprising a nucleotide sequence encoding a herbicide resistant target gene and the corresponding promoter and 3′ end sequences directing expression in the source from which sequences were isolated.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene or recombinant DNA construct that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing, stability, or translation of the associated coding sequence. Regulatory sequences may include, but are not limited to, promoters, translation leader sequences, introns, and polyadenylation recognition sequences.

A polynucleotide sequence encoding a “portion” of a gene or coding sequence is a polynucleotide sequence encoding at least 10 amino acids and capable of producing an active fructosyltransferase in an embryo cell.

“Promoter” refers to a DNA sequence capable of controlling the expression of a coding sequence or functional RNA. The promoter sequence consists of proximal and more distal elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of some variation may have identical promoter activity.

As used herein, “substantially similar” refers to polynucleotides, genes, coding sequences, and the like, wherein changes in one or more nucleotide bases results in substitution of one or more amino acids, but do not affect the functional properties of the polypeptide encoded by the nucleotide sequence. “Substantially similar” also refers to polynucleotides wherein changes in one or more nucleotide bases does not affect the ability of the polynucleotide to mediate alteration of gene expression by gene silencing through for example antisense or co-suppression technology. “Substantially similar” also refers to modifications of the polynucleotide of the instant invention such as deletion or insertion of one or more nucleotides that do not substantially affect the functional properties of the resulting transcript vis-a-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

Substantially similar polynucleotides may be selected by screening polynucleotides representing subfragments or modifications of the polynucleotides of the instant invention, wherein one or more nucleotides are substituted, deleted and/or inserted, for their ability to affect the level of the polypeptide encoded by the unmodified polynucleotides in a plant or plant cell. For example, a substantially similar polynucleotides representing at least one of 30 contiguous nucleotides derived from the instant polynucleotides can be constructed and introduced into a plant or plant cell. The level of the polypeptide encoded by the unmodified polynucleotides present in a plant or plant cell exposed to substantially similar polynucleotide can then be compared to the level of the polypeptide in a plant or plant cell that is not exposed to the substantially similar polynucleotides.

An “intron” is an intervening sequence in a gene that does not encode a portion of the protein sequence. Thus, such sequences are transcribed into RNA but are then excised and are not translated. The term is also used for the excised RNA sequences. An “exon” is a portion of the sequence of a gene that is transcribed and is found in the mature messenger RNA derived from the gene, but is not necessarily a part of the sequence that encodes the final gene product.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase 1. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

“Transformation” refers to the transfer of a nucleic acid fragment into the genome of a host organism. Host organisms containing the transformed nucleic acid fragments are referred to as “transgenic” organisms. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. The preferred method of cell transformation of plant cells is the use of particle-accelerated or “gene gun” transformation technology (Klein et al., (1987) Nature (London) 327:70-73; U.S. Pat. No. 4,945,050), or an Agrobacterium-mediated method using an appropriate Ti plasmid containing the transgene (Ishida Y. et al., 1996, Nature Biotech. 14:745-750).

“Insert,” “transfer,” “introduce,” and the like refer to the action of using a nucleic acid fragment in the process of transformation.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”). “PCR” amplification or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

Invention

The present invention includes a plant and plant part comprising at least one recombinant DNA molecule comprising an embryo specific promoter operably linked to at least a portion of at least one coding sequence for a plant fructosyltransferase, operably linked to a vacuole targeting sequence, said molecule sufficient to express a protein capable of producing fructan having a degree of polymerization of at least three, in an embryo of said plant, or any progeny thereof, wherein said progeny comprise said molecule.

In accordance with the present invention, a plant includes and is not limited to a plant, expressing a protein capable of producing fructan having a DP of at least three in the embryo. Such fructan producing plants include dicots and monocots. Dicots include and are not limited to legumes, including soybean, and the like. Monocots include and are not limited to cereals, also known as grasses, including and are not limited to corn and the like, for example.

Also within the scope of the invention are plant parts obtained from such plants. Plant parts include differentiated and undifferentiated tissues, including but not limited to, embryos, roots, stems, shoots, leaves, pollen, seeds, grains, tumor tissue, and various forms of cells and culture such as and not limited to single cells, protoplasts, embryos, and callus tissue. The plant tissue may be in plant, organ, tissue or cell culture. Grain and seed are used interchangeably herein. In addition, a corn kernel is a grain.

The term “corn” refers to Zea mays, and is used herein interchangeably with maize. The term “soybean” refers to Glycine max. The term “Jerusalem artichoke” refers to Helianthus tuberosus, Term “guayule” refers to Parthenium argentatum. The term “chicory” refers to Cichorium intybus. The term “tomato” refers to Lycopersicon esculentum.

In accordance with the present invention, plant sources may be the plant per se. In addition, the plant source of the subject invention includes and is not limited to the seed, grain, plant cells, plant protoplasts, plant cell tissue culture from which plants may be regenerated, plant calli, plant clumps, and plant cells that are intact in plants or parts of plants such as embryos, pollen, flowers, ears, cobs, leaves, husks, stalks, roots, root tips, anthers, silk, and the like.

As used herein, “embryo” refers to the embryo axis and cotyledons in dicots and the embryo axis and scutellum in monocots. “Embryo specific promoter” refers to a promoter which is expressed throughout the embryo axis, the cotyledons, or the embryo axis and coytledons in dicots and embryo axis, the scutellum, or the embryo axis and scutellum in monocots. Preferred embryo-specific promoters are seed protein promoters, which may be expressed in the cotyledons or the cotyledons and embryo axis.

To date, a multitude of embryo-specific promoters are known which direct strong seed-specific expression of a transgene or recombinant DNA construct. Examples include, but are not restricted to, the Kunitz trypsin inhibitor (KTi) promoter (Jofuku et al. (1989) Plant Cell 1:1079-1093; Perez-Grau, L. and Goldberg, R. (1989) Plant Cell 1:1095-1109), the Phaseolin promoter (Burow, M. D. et. al. (1992) Plant J. 2:537-548), the promoter of the gene for the α′-subunit of the β-conglycinin (Beachy, R. N. et al. (1985) EMBO J. 4:3047-3053; Harada, J. J. et. al. (1989) Plant Cell 1: 415-425), the soybean 2S-albumin promoter (Coughlan, S. J. and Winfrey, R. J, U.S. Pat. No. 6,177,613 issued January 2001), the soybean Glycinin promoter (Nielsen, N. C. et., al. (1989) Plant Cell 1:313-328), the maize (Zea mays L.) globulin-1 promoter (Belanger F. C. and Kriz A. L.(1991) Genetics 129:863-872), the maize oleosin promoter (Lee, K. and Huang, A. H. (1994) Plant Mol. Biol. 26:1981-1987).

In accordance with the present invention, “vacuole targeting sequence,” also referred to as vacuole sorting signals (BMBP, page 192) refers to a sequence that after translation directs a gene product, polypeptide, protein, or the like to a vacuole. Vacuole targeting sequences are known in the art and are operably linked to the other parts of the recombinant DNA molecule (BMBP, pages 192-193).

“Fructosyltransferase” refers to a protein coded for by any one of several genes having the property of producing a carbohydrate polymer consisting of repeating fructose residues. Fructosyltransferases may be isolated from a plant or bacterial source. The repeating fructose residues may be linked by β2-1 linkage, a p2-6 linkage, or any combination of the two types of linkages. The polymer of repeating fructose residues may contain one terminal glucose residue, derived from a sucrose molecule, and at least two fructose residues. The polymer of repeating fructose residues in any form, with any combination of linkages, and with any number of fructose residues, is referred to generally as a “fructan”. Fructosyltransferases include and are not limited to fructose:fructose fructosyltransferase and sucrose:sucrose fructosyltransferase.

A “fructosyltransferase gene” or “ftf” refers to the polynucleotide coding for a fructosyltransferase protein. “FTF” refers to fructosyltransferase protein or fructosyltransferase protein activity. The term “deleterious effect” as used herein, refers to a direct or indirect injurious effect on a plant or plant cell as a result of fructan accumulation, such that the plant or plant cell is prevented from performing certain functions including, but not limited to, synthesis and transport of carbohydrates within a cell and throughout the plant, regeneration of transgenic plants or tissue, development of the plant or plant cell to maturity, or the ability to pass the desired trait or traits to progeny. For purposes of the present invention, frutosyltransferases and coding sequences therefor may be isolated from plant or bacterial sources. Plants are the preferred source of fructosyltransferase coding sequences. Such plant sources include and are not limited to Jerusalem artichoke and guayule.

“Fructan” refers to any compound in which one or more fructosyl-fructose linkages constitute a majority of the linkages (the presence of a glucose unit is optional).

“Fructose” refers to a very sweet sugar, C₆H₁₂O₆, occurring in many fruits and honey and used as a preservative for foodstuffs and as an intravenous nutrient. Fructose is also known as fruit sugar, levulose.

A “fructosyl unit” refers to a fructose molecule linked to another sugar molecule (e.g. glucose, fructose, galactose, mannose).

“Inulin” refers to fructan that has mostly β-2,1 fructosyl-fructose linkages (the presence of a glucose unit is optional). “Degree of polymerization” or “DP” refers to the number of fructose residues contained in an individual fructan polymer. DP varies greatly depending on the source from which the fructan is isolated. For purposes of the present invention, a transgenic plant should be capable of producing fructan having a degree of polymerization of at least three. Thus, a plant embryo comprising a coding sequence for fructosyltransferase in accordance with the present invention produces fructan having a degree of polymerization of at least three.

In accordance with the present invention, a monocot embryo comprising a transgene for sucrose:sucrose fructosyltransferase, as well as a grain (corn kernels for example) containing a monocot embryo, contain fructan exhibiting a degree of polymerization of at least three and includes fructan having a degree of polymerization of at least four, at least five, at least six, and at least seven. A monocot comprising a transgene for sucrose:sucrose fructosyltransferase and fructose:fructose fructosyltransferase, contains in the embryo or grain containing the embryo fructan having a degree of polymerization of at least three, and also includes fructan having degrees of polymerization of at least four, at least five, at least six, at least seven, at least eight, at least nine and at least ten. Fructan having a degree of polymerization of up to about 200 may be obtained from plants.

A dicot embryo transformed with a coding sequence for sucrose:sucrose fructosyltransferase and fructose:fructose fructosyltransferase produces fructan having a degree of polymerization of at least three, and includes fructan having degrees of polymerization of four and five.

It is expected that a dicot embryo transformed with a coding sequence for sucrose:sucrose fructosyltransferase will also produce fructan with a degree of polymerization of at least three. In the same manner, progeny thereof are expected to produce fructan having a degree of polymerization of at least three.

In accordance with the present invention, the plant cell may be transformed by at least one recombinant DNA molecule that results in production of fructan having a degree of polymerization of at least three. Such recombinant DNA molecule includes and is not limited to a recombinant DNA molecule encoding at least a portion of a coding sequence for a plant fructosyltransferase, wherein the fructosyltransferase is sucrose:sucrose fructosyltransferase, and a recombinant DNA molecule encoding at least a portion of a coding sequence for a eukaryotic fructosyltransferase, wherein the fructosyltransferase is sucrose:sucrose fructosyltransferase and fructose:fructose fructosyltransferase.

Also included as recombinant DNA molecule is a recombinant DNA molecule comprising sucrose:sucrose fructosyltransferase and fructose:fructose fructosyltransferase. Yet another recombinant DNA molecule is a first recombinant DNA molecule and a second recombinant DNA molecule, wherein the first DNA molecule comprises a coding sequence for sucrose:sucrose fructosyltransferase and the second DNA molecule comprises a coding sequence for fructose:fructose fructosyltransferase.

The transformed plant is then grown under conditions suitable for the expression of the recombinant DNA molecule. Expression of the recombinant DNA molecule results in fructan having a degree of polymerization of at least three.

The present invention is also directed to a method of producing fructan in a plant comprising constructing at least one recombinant DNA molecule comprising an embryo specific promoter operably linked to a vacuole targeting sequence operably linked to at least one coding sequence for a fructosyltransferase, transforming a plant with the construct, regenerating the plant to produce seed, harvesting seed from the plant, and extracting fructan from the harvested seed.

The regenerated plant may be multiplied to obtain a useful amount of seed that may be employed in large scale growth, such as farming, of crops from which fructan may be obtained. In addition, grain per se, comprising a transgene for a fructosyltransferase, is useful as feed for animals.

The present invention also includes a method of screening transgenic plant tissue expressing embryo targeted genes comprising preparing Type-II maize callus for transformation, transforming callus, selecting transgenic callus lines, regenerating transgenic somatic embryos, and propagating transgenic somatic embryos for plant production and early trait analyses.

Another embodiment of the present invention is a foodstuff comprising fructan produced by a plant comprising at least one recombinant DNA molecule comprising an embryo specific promoter operably linked to a vacuole targeting sequence operably linked to at least one coding sequence for a fructosyl-transferase, the molecule sufficient to express fructan of at least DP3 in a grain of the plant, or any progeny thereof, wherein the progeny comprise said molecule. The fructan of such plant may be inulin.

Industrial products comprising fructan produced in accordance with the present invention are also included herein. Such industrial products include and are not limited to a hydrocolloid, a bleach activator, a dispersing agent, glue, and a biodegradable complexing agent.

Also, within the scope of this invention are food and beverages which have incorporated therein a fructan product of the invention. The beverage can be in a liquid or a dry powdered form.

“Foodstuff,” including “food” and “feed,” is used herein to mean substances for consumption that contain fructan or grain from the plant of the present invention. Grain, for example, is useful in such food and feed, for humans and animals, respectively.

The foods to which fructan of the invention can be incorporated/added include almost all foods/beverages. For example, there can be mentioned meats such as ground meats, emulsified meats, marinated meats, and meats injected with a product of the invention; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; and salad dressings; and dips and spreads such as mayonnaise and chip dips. Fructan can be added in an amount selected to deliver a desired dose to the consumer of the food and/or beverage.

Depending on their origin, fructan vary greatly in size and functionality allowing for the use of fructan in a wide variety of commercial applications. Fructan with a low DP have a sweet taste while fructan with a higher DP provide better functionality and a texture similar to fat. The food industry uses fructan as low calorie replacements because the human body is not capable of metabolizing them. Furthermore, the food industry uses fructan to make functional and healthier foods and food additives. This health effect is based on the observation that fructan, which reach the colon intact, are fermented resulting in prebiotic effects towards certain beneficial species of Bifidobacteria and advantageous effects promoting overall health. These health effects include improvement of intestinal microflora, protection against intestinal infections, prevention of constipation, reduction of serum cholesterol, increased mineral absorption, anti-colon-cancer effects and increased production of B-vitamins (Information pamphlet of Imperial-Sensus, Sugar Land, Tex. 77487). The feed industry also takes advantage of animals being incapable of metabolizing fructan. Thus, the addition of fructan to feed enhances animal health and performance through selective fermentation by beneficial organisms such as Bifidibacteria at the expense of pathogenic organisms such as E. coli and Salmonella. This selective fermentation leads to altered fatty acid profiles, increased nutrient absorption, and decreased levels of blood cholesterol in the animal. Fructan is also considered to be an excellent source of fructose for the production of high-fructose syrup. Fructose may be obtained by the hydrolysis of fructan into individual fructose residues. This process for the preparation of fructan has a tremendous advantage over the current, technically demanding, process of enzymatically converting starch into high fructose syrup. Using fructan as the starting material would, therefore, significantly reduce production costs. Fructan with a medium to high DP are useful for industrial applications, such as the production of biodegradable complexing agents for heavy metals, biodegradable glues, filler/binders and surfactants.

The most commonly used fructan to date is inulin, which is commercially obtained by extraction of plants or plant parts. Inulin is a polydisperse carbohydrate built up of fructose units, with an optional glucose unit, that cannot be digested by the human digestive enzymes and reaches the colon intact. In addition to inducing a health benefit in humans and animals, inulin has some nutritional as well as functional benefits that result in advantageous qualities in food and feed. The nutritional benefits are mainly found in the fact that inulin is a soluble dietary fiber, has a low caloric value, and is suitable for diabetics. The functional benefits of inulin include, in part, its function as a water soluble compound, texturizer, taste improver, good solubility, sugar and fat replacer, fiber enrichment, and use in filler/binder for tablets. Given the inulin benefits mentioned above, inulin has been used in the manufacture of a wide variety of food and feed products as well as drinks and non-food products. Depending on the application, inulins with a different profile are used. Inulins of DP2 to DP7, also referred to as oligofructose, are commonly used as low caloric sweeteners. Low DP inulins as well as inulins with an average DP of 9 are also used as a soluble dietary fiber and as an ingredient in food and feed products emphasizing health benefits. Inulins with an average DP of 10 and average DP of >23 are commercially available (Orafti, Tienen, Belgium) and are mainly used in food and feed products for their functional benefits described above.

Commercial use of fructan is currently severely limited due to the high cost and low acreage of production. Fructan used in low-calorie foods are currently extracted from chicory (Cichorium intybus) and Jerusalem artichoke (Helianthus tuberosus). Larger polymers synthesized by bacteria are not currently produced on a commercial scale. Chicory and Jerusalem artichoke are cultivated mainly in Europe and using non-economic farming practices. A few crops adapted to wide growing regions, such as oat, wheat, and barley, accumulate fructan and only at extremely low levels.

The disclosure of each reference set forth in this application is incorporated herein by reference in its entirety.

### EXAMPLES

The present invention is further illustrated in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions. Thus, various modifications of the invention in addition to those shown and described herein will be apparent to those skilled in the art from the foregoing description. Such modifications are also intended to fall within the scope of the appended claims.

### Example 1

### Method For Early Screening of Embryo-Targeted Traits in Transgenic Maize

A method for early screening of embryo-targeted traits in transgenic maize using transgenic somatic embryos was developed. The method consists of:

1) preparing Type-II maize callus for transformation,

2) transforming callus using the particle bombardment technique,

3) selecting transgenic callus lines,

4) regenerating transgenic somatic embryos,

5) propagating transgenic somatic embryos for early trait analyses and plant production, and.

6) analyzing somatic embryos for phenotypic trait.

1. Preparation of Callus for Transformation

A rapidly growing Type-II maize callus is transferred to #4 Whatman filter paper placed on a modified Chu (N6) callus maintenance medium (Chu, C. C., et al. (1975) Scientia Sinic. 18:659). The callus is spread in a thin layer covering the filter paper in a circular area of approximately 4 cm in diameter, the filter paper is transferred to a petri dish, and is incubated in the dark in a growth chamber (45% humidity, 27-28° C.) for two to four days before transformation via gold particle bombardment. On the day of bombardment, the callus-containing filter is transferred to a petri dish containing modified Chu (N6) high osmoticum medium, wrapped with parafilm, and placed in the dark growth chamber for four additional hours. Just prior to bombardment, the petri dishes are left partially ajar for thirty minutes in the laminar flow hood to allow moisture on the tissue to dissipate.

2. DNA/Gold Preparation and Particle Bombardment Procedure

DNA is precipitated onto gold particles and the corn callus is bombarded with DNA/gold according to the method of Fromm et al. (Fromm et al. (1990) Biotechnology (NY) 8:833-839).

3. Selection of Transgenic Maize Callus Lines

Transgenic maize callus lines are selected by transferring the filter paper containing the callus through different media as follows:

Transfer 1: Within 60 minutes following bombardment, callus-containing filter papers are placed onto fresh callus maintenance medium, wrapped with parafilm, and incubated in the dark chamber for 3-4 days.

Transfer 2: After 3-4 days, plates containing filters with bombarded callus are checked for contamination and 3-4 mm clumps of callus are subcultured onto selection medium which is a modified Chu (N6) medium supplemented with 2-10 ppm bialaphos. Plates containing the newly subcultured callus on selection medium are wrapped with parafilm and incubated in the dark.

Transfer 3: After about 7-14 days (depending on growth rate) larger clumps are split into several smaller pieces, keeping track of all pieces originating from each original clump, and subcultured onto fresh selection medium, as above.

Transfer 4: After another ˜14 days all callus are transferred onto fresh selection medium containing bialaphos, keeping track of the lineage of each piece as above. If needed, clumps may again be split into several pieces at this transfer.

Transfer 5: After 2 or 3 weeks, callus may be transferred onto fresh selection medium, keeping track of unique lines as above. This depends on the growth of the tissue and the experiment. Approximately 2-3 weeks after transfers 4 or 5, bialaphos-tolerant, rapidly-growing callii (transformation events) are identified and individually subcultured onto fresh selection. Callii are incubated in this medium for another two-weeks.

4. Regeneration of Transgenic Somatic Embryos

Transgenic callus events are isolated onto plates of fresh selection medium, one to four independent callus events per plate. After two weeks, each event is assigned a number, sampled for PCR analysis, placed in an individual plate containing a modified MS medium (Murashige, T. and Skoog, F. (1962) Physiol. Plant. 15:473), and grown in the dark for 10-14 days. This step is the first stage of regeneration to plants through somatic embryogenesis. During this time, the embryogenic callus grows to form many discrete, hard, white somatic embryos.

5. Propagation of Somatic Embryos for analysis and Regeneration of Transgenic Plants

After 10-14 days in the dark on first-stage regeneration medium, some of the hard, white somatic embryos are used for analyses and at the same time some are regenerated into plants. For analysis the somatic embryos may be transferred to empty plastic sample dishes and analyzed immediately, may be transferred to empty plastic sample dishes and frozen immediately at −78° C. until analyzed, or may be transferred onto second-stage regeneration medium (a modified MS medium, in which the concentration of MS salts is reduced to one-half the concentration normally used (Murashige, T. and Skoog, F. (1962) Physiol. Plant 15: 473) for transport and later analysis. For regeneration into plants, the hard, white somatic embryos are transferred onto second-stage regeneration medium and placed in the light at 26° C.

6. Analysis of Somatic Embryos for Transgenic Phenotypic Trait

Somatic embryos are ground to homogeneity and analyzed for phenotypic trait such as protein, oils, carbohydrates (such as in Examples 5 and 9), isoflavones, flavones, etc.

### Example 2

### Construction of a Cassette for Embryo-Targeted Expression of Jerusalem Artichoke SST in Transgenic Zea mays L.

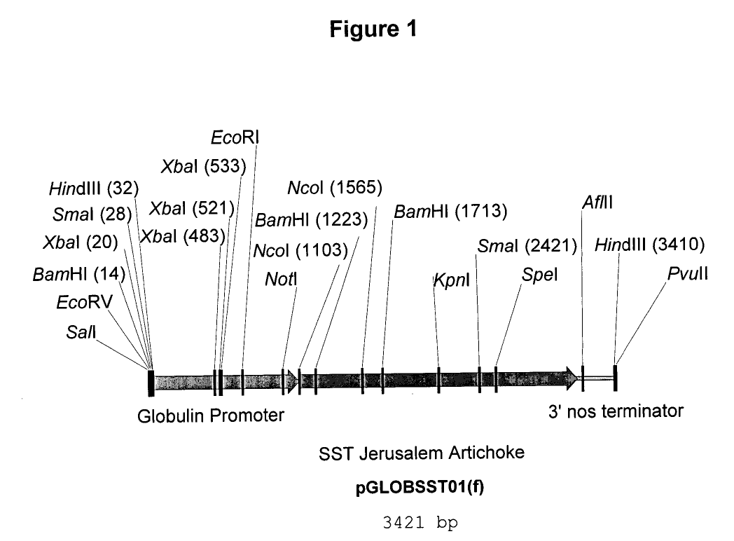

A cassette designed for the embryo-specific expression in maize of the Jerusalem artichoke sucrose:sucrose fructosyltransferase (SST) was assembled. This cassette, GLOBSST01(f) is shown in FIG. 1 contains a maize embryo-specific globulin promoter directing translation of the entire Jerusalem artichoke SST coding region followed by a 3′ nos termination signal.

The GLOBSST01(f) cassette was assembled into plasmid vector pGLOBSST01 by replacing the maize endosperm-specific 10 kD zein promoter in plasmid 10 kD-SST-17 with the maize embryo-specific globulin promoter. Plasmid 10 kD-SST-17 (described in PCT publication No. WO99/46395, published Sep. 16, 1999) contains the 10 kD zein promoter directing the expression of the Jerusalem artichoke SST, including native and secretory vacuolar signals. To assemble plasmid 10 kD-SST-17 an intermediary plasmid was assembled by removing the polynucleotide fragment encoding SacB from plasmid pCyt-SacB (described by Caimi et al. (1996) Plant Physiol. 110:355-363) by digesting with Nco I and Hind III and inserting the polynucleotide fragment encoding the Jerusalem artichoke SST that had been removed from plasmid pSST403 (described in PCT publication WO 96/21023, published Jul. 11, 1996) by digestion with Nco I and Hind III. The polynucleotide fragment comprising the 10 kD zein promoter and SST coding region was removed from this intermediary plasmid by digestion with Sma I and Bam HI. The 10 kD-SST fragment was then inserted into Sma I and Bam HI-digested plasmid pKS17 to form plasmid 10 kD-SST-17. Plasmid pKS17 was derived from the commercially-available plasmid pSP72 (Promega Biotech, Madison, Wis.) by deleting from pSP72 the polynucleotide fragment corresponding to the beta lactamase coding region (nucleotides 1135 through 1995) and inserting between the E. coli T7 RNA polymerase promoter and termination signal a polynucleotide fragment encoding HPT. The polynucleotide fragment encoding HPT corresponds to the polynucleotide fragment from E. coli strain W677 encoding hygromycin B phosphotransferase which, when under the control of a bacterial promoter, allows for selection of transformed cells in certain bacteria (Gritz, L. and Davies, J. (1983) Gene 25:179-188). Finally, the embryo-specific globulin promoter described in U.S. Pat. No. 5,773,691 was used to replace the 10 kD zein endosperm-specific promoter in plasmid 10 kD-SST-17. To do this, first, an Nco I restriction endonuclease site present in the globulin promoter in plasmid pCC50 was destroyed to form plasmid pBT747. Then, the polynucleotide fragment containing the sequences for the globulin promoter were removed from plasmid pBT747 by digestion with Sal I and Nco I and the fragment containing the globulin promoter was used to replace the 10 kD zein promoter in plasmid 10 kD-SST-17 to create plasmid pGLOBSST01. The sequence of plasmid pGLOBSST01 is shown in SEQ ID NO:1.

Digestion of pGLOBSST01 with Hind III yields a 3378 bp DNA fragment containing the SST coding region surrounded by the embryo-specific globulin promoter and the nos 3′ transcription termination region. This fragment was designated GLOBSST01(f), is shown in FIG. 1, and contains the complete embryo-specific SST expression cassette. The GLOBSST01(f) DNA fragment was purified by gel electrophoresis and was used for transformation into corn by particle bombardment as described below.

### Example 3

### Construction of a Cassette for Embryo-Targeted Expression of Jerusalem Artichoke FFT in Transgenic Zea mays L.

A cassette designed for the embryo-specific expression in maize of the Jerusalem artichoke fructan:fructan fructosyltransferase (FFT) was assembled. This cassette, GLOBFFT01(f), contains a maize embryo-specific globulin promoter directing translation of the entire Jerusalem artichoke FFT coding region followed by a 3′ nos termination signal.

The GLOBFFT01(f) cassette was assembled into plasmid pGLOBFFT01 by replacing the maize endosperm-specific 10 kD zein promoter in plasmid 10 kD-FFT-17 with the maize embryo-specific globulin promoter. Plasmid 10 kD-FFT-17 (described in PCT publication No. WO99/46395, published Sep. 16, 1999) contains the 10 kD zein promoter directing the expression of the Jerusalem artichoke FFT, including native and secretory vacuolar signals. To assemble plasmid 10 kD-FFT-17 an intermediary plasmid was constructed by removing the polynucleotide fragment encoding SacB from plasmid pCyt-SacB (described by Caimi et al. (1996) Plant Physiol. 110:355-363) by digestion with Nco I and Bam HI and replacing this fragment with the polynucleotide fragment encoding the Jerusalem artichoke FFT from plasmid pSST405 (described in PCT publication WO 96/21023, published Jul. 11, 1996). The polynucleotide fragment containing the 10 kD zein promoter and the FFT coding region was removed from this intermediary plasmid by digestion with Sma I and Sal I. The 10 kD-FFT fragment was inserted into plasmid pKS17 (described in Example 2) that had been digested with Sma I and Bam HI to form plasmid 10 kD-FFT-17. Finally, the embryo-specific globulin promoter was removed from plasmid pBT747 (described in Example 3) by digesting with Sma I and Nco I and used to replace the 10 kD zein endosperm-specific promoter in plasmid 10 kD-FFT-17 to create plasmid pGLOBFFT01. The sequence of plasmid pGLOBFFT01 is shown in SEQ ID NO:2.

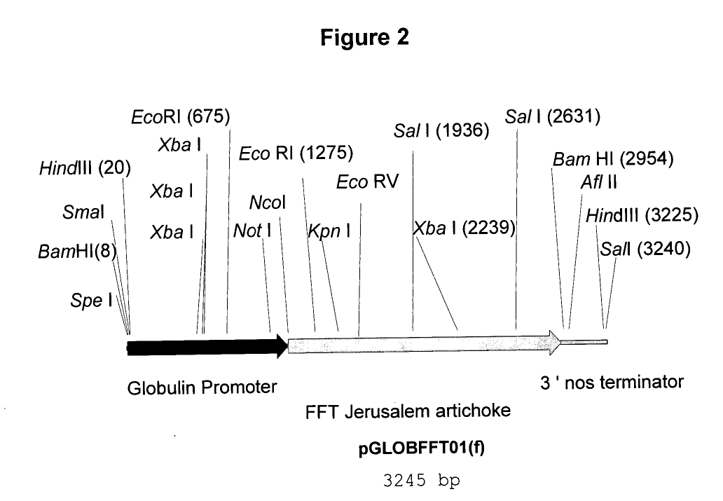

Digestion of pGLOBFFT01 with Hind III yields a 3344 bp DNA fragment, containing the FFT coding region surrounded by the embryo-specific globulin promoter and the nos 3′end. This fragment was designated GLOBFFT01(f), is depicted in FIG. 2, and contains the complete embryo-specific FFT expression cassette. This fragment was purified by gel electrophoresis and was used for transformation into corn by particle bombardment as described below.

### Example 4

### Transformation of Corn Embryos with Embryo-Targeted SST. FFT, or Both and Detection of the Transgenes

Transformation of Corn Embryogenic Calli

For corn embryogenic calli transformation, the purified DNA fragments containing the embryo-specific cassettes were co-bombarded with pDetric, a polynucleotide fragment containing the bar gene under the control of the CaMV 35S promoter and OCS 3′-end. The bar gene (Murakami et al. (1986) Mol. Gen. Genet 205:42-50; DeBlock et al. (1987) EMBO J. 6:2513-2518) encodes phosphinothricin acetyl transferase (PAT) which confers resistance to herbicidal glutamine synthetase inhibitors such as phosphinothricin (bialophos). The sequence of the Hind III polynucleotide fragment corresponding to pDETRIC is shown in SEQ ID NO:3. Other selectable markers may be used in the invention such as, and not limited to, pALSLUC (Fromm, et al, (1990) Biotechnology 8:833-839) that contains polynucleotides encoding a mutant acetolactate synthase (ALS) that confers resistance to chlorsulfuron under the control of the CaMV 35S promoter.

Embryogenic maize callus derived from crosses of the inbred lines Al 88 and B73 (Armstrong et al.(1991) Maize Genetics Cooperation Newsletter 65:92-93) were co-transformed with pDetric and pGLOBSST01(f), or with pDetric, pGLOBSST01(f), and pGLOBFFT01(f) using microprojectile bombardment (Klein T. M. et. al. (1987) Nature 327:70-73).

Transformed embryogenic cells were recovered on medium containing glufosinate-ammonium. Transgenic embryos selected as in Example 1 were analyzed for production of fructan or transferred to 12 inch pots containing METROMIX™ soil (Scotts-Sierra Company, Marysville, Ohio) and grown to maturity in the greenhouse

Detection of SST and FFT Transgenes

The presence of the SST and FFT in transgenic embryos or plants was accomplished by PCR analyses of RNA obtained from leaf tissue.

Oligonucleotide primers SST-1 (SEQ ID NO:4) and SST-2 (SEQ ID NO:5) were used to detect the polynucleotide fragment encoding Jerusalem artichoke SST. Oligonucleotide primers FFT-1 (SEQ ID NO:6) and FFT-2 (SEQ ID NO:7) were used to detect the polynucleotide fragment encoding Jerusalem artichoke FFT.

| | |
|---|---|
| SST-1: | |
| 5′- TTCGTAACTCAGTTGCCAAATATTG-3′ | (SEQ ID NO:4) |
| SST-2: | |
| 5′- CCAGCCCGTTTGTGTGTACGGT-3′ | (SEQ ID NO:5) |
| FFT-1: | |
| 5′- GTTCGTATCGTCACCAATTCG-3′ | (SEQ ID NO:6) |
| FFT-2: | |
| 5′- GTGCACTATCATTGGTTAACG-3′ | (SEQ ID NO:7) |

After amplification and separation of the DNA fragments by polyacrylamide gel electrophoresis, transgenic maize somatic embryos or transgenic plants were identified that contained only the Jerusalem artichoke SST, only the Jerusalem artichoke FFT, or both, the Jerusalem artichoke SST and the Jerusalem artichoke FFT.

### Example 5

### Corn Fructan Composition Analyses

The carbohydrate composition of transgenic somatic embryos or transgenic plants identified in Example 4 as containing the GLOBFFT01(f) and/or GLOBSST01(f) cassettes was measured by high performance anion exchange chromatography/pulsed amperometric detection (HPAE/PAD). Individual seeds from transgenic lines were harvested at 35-50 days post-pollination (DPP) for detection of carbohydrate composition. The seeds were frozen in liquid nitrogen, ground with a mortar and pestle, and transferred to 15 mL microcentrifuge tubes. Fresh individual somatic embryos were rapidly washed in water, dried on a paper towel, and transferred into 1.5 mL microcentrifuge tubes. Ethanol (80%) was added to the tubes and the samples were heated to 70° C. for 15 minutes. The samples in the 15 mL tubes were centrifuged at 4, 000 rpm and the samples in the 1.5 mL tubes were centrifuged at 14,000 rpm for 5 minutes at 4° C. and the supernatant collected. The pellet was re-extracted two additional times with 80% ethanol at 70° C. The supernatants were combined, dried down in a speedvac, and the pellet re-suspended in water.

For HPAE analysis, the extracts were filtered through a 0.2 μm Nylon-66 filter (Rainin, Emeryville, Calif.) and analyzed by HPAE/PAD using a DX500 anion exchange analyzer (Dionex, Sunnyvale, Calif.) equipped with a 250×4 mm CarboPac PA1 anion exchange column and a 25×4 mm CarboPac PA guard column. Soluble carbohydrates and inulin were separated with a 30 minute linear gradient of 0.5 to 170 mM NaAc in 150 mM NaOH at a flow rate of 1.0 mL/min. A mixture of 20 mg/L of glucose, fructose, sucrose, raffinose, stachyose, 1-kestose (DP3), 1-kestotetraose (DP4), and 1-kestopentaose (DP5, Megazyme, Bray, Ireland) was used as a standard.

Soluble sugars as well as 1-kestose, 1-kestotetraose, and 1-kestopentaose were quantified by comparison to standards using HPAE/PAD. To quantify inulin, the fructan molecules were hydrolyzed with 150 mM HCl and incubated at 60° C. for up to 60 minutes. This solution was neutralized by addition of NaOH and the released fructose was quantified using HPAE/PAD. In the present application fructan is expressed in μmol hexose equivalent/g fresh weight (μmol/g f w).

Carbohydrate Analysis of Transgenic Maize Somatic Embryos

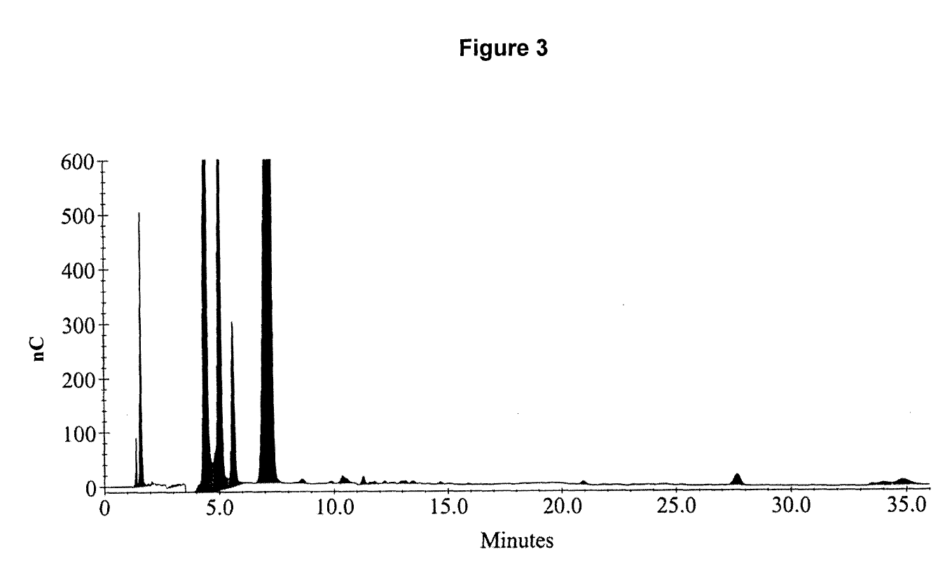

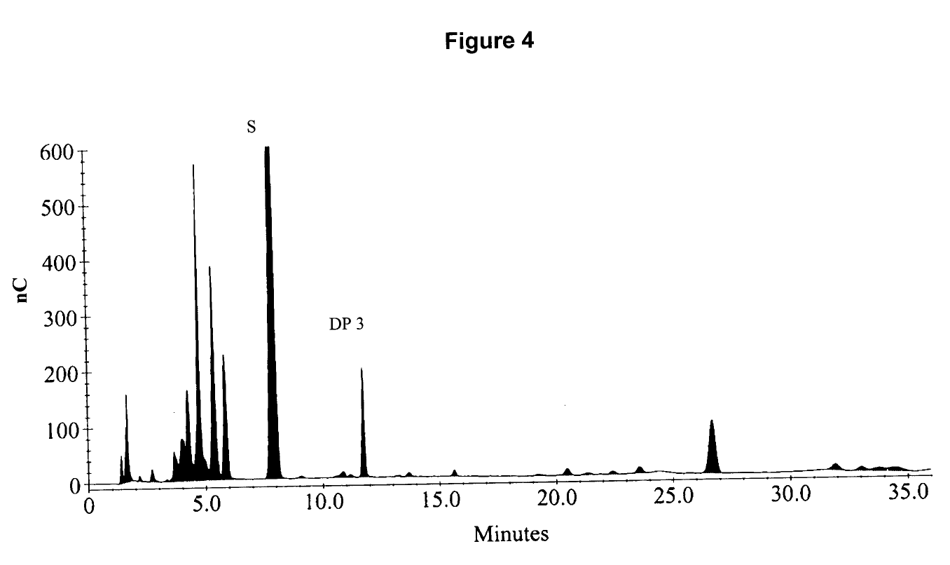

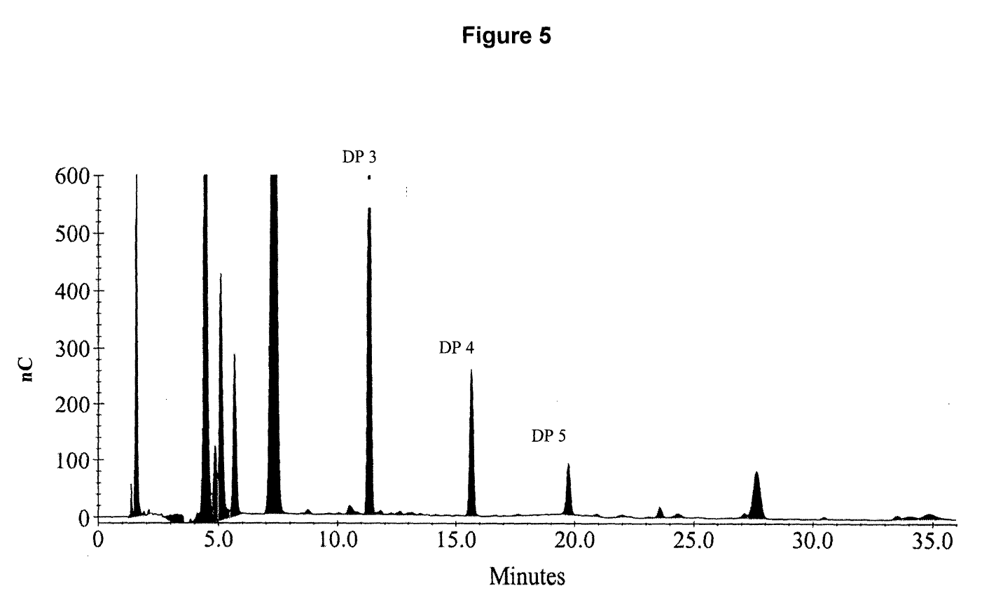

A carbohydrate profile resulting from HPAE/PAD analysis of maize somatic embryos not expressing the GLOBSST01(f) or GLOBFFT01(f) cassettes is shown in FIG. 3. A carbohydrate profile resulting from HPAE/PAD analysis of transgenic maize somatic embryos expressing intact copies of the GLOBSST01(f) cassette is shown in FIG. 4, and resulting from transgenic maize somatic embryos expressing GLOBSST01(f) and GLOBFFT01(f) cassettes is shown in FIG. 5.

The carbohydrate profile in FIG. 3 shows that inulin is not detected in maize somatic embryos not expressing the GLOBSST01(f) or GLOBSST01(f) cassettes. The carbohydrate profile in FIG. 4 shows that transgenic maize somatic embryos expressing the GLOBSST01(f) cassette accumulated inulin-type fructose polymers of DP3 and DP4 and in FIG. 5 shows that transgenic maize somatic embryos expressing both, GLOBSST01(f) and GLOBFFT01(f), cassettes accumulated inulin-type fructose polymers of DP3 through DP7.

Inulin-accumulating embryos were allowed to develop into plants using standard tissue culture techniques.

Carbohydrate Analysis of Transgenic Maize Seeds

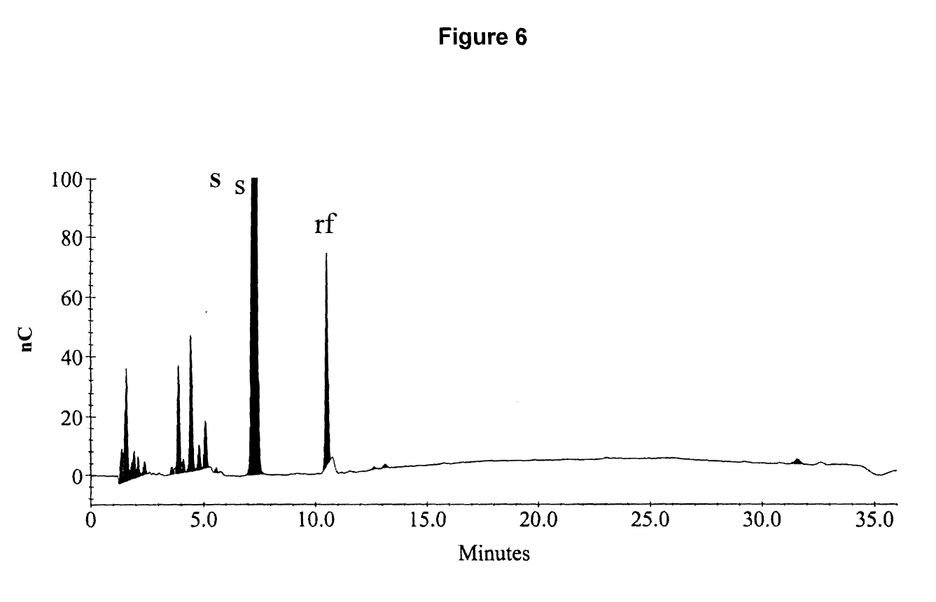

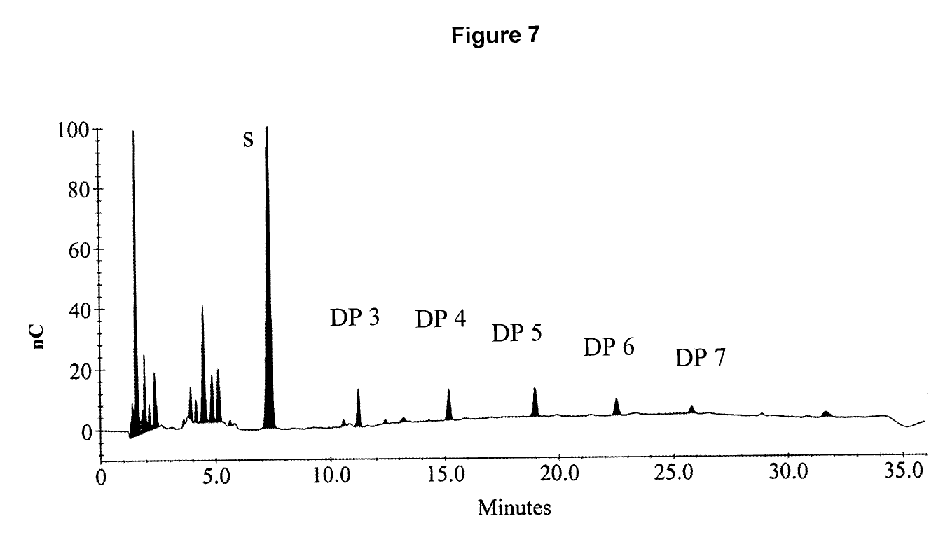

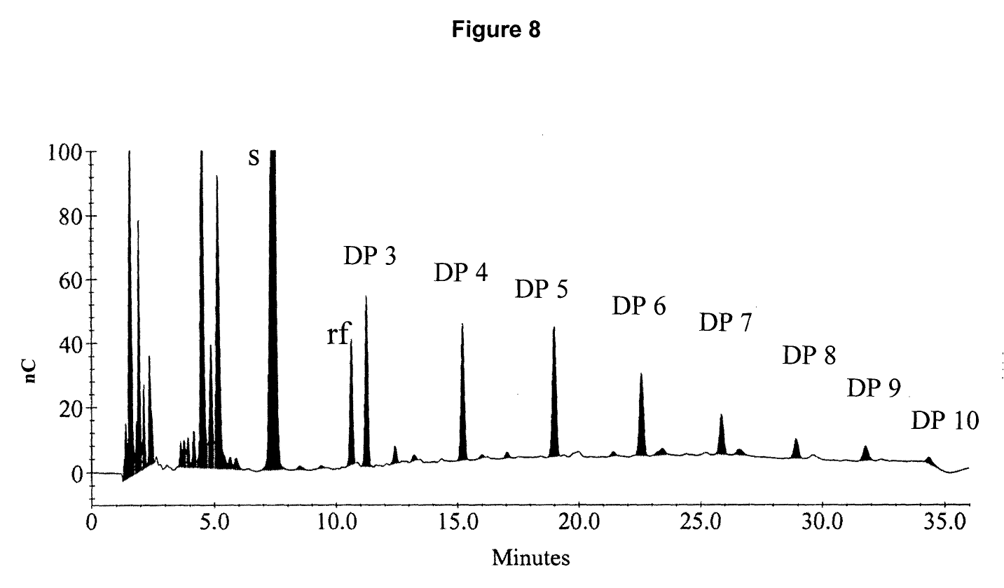

Individual mature kernels were obtained from transgenic plants of all events. The kernels were frozen in liquid nitrogen and processed for analyses as indicated above. The carbohydrate profile resulting from HPAE/PAD analysis of kernels from transgenic maize plants not containing GLOBSST01(f) or GLOBFFT01(f) cassettes are shown in FIG. 6. Carbohydrate profiles of kernels from transgenic maize plants containing intact copies of the GLOBSST01(f) cassette are shown in FIG. 7 and of kernels from transgenic maize plants expressing, both, GLOBSST01(f) and GLOBSST01(f), cassettes are shown in FIG. 8.

No inulin was detected in kernels from transgenic plants not containing the GLOBSST01(f) or the GLOBFFT1(f) cassettes (FIG. 6). Kernels from transgenic plants expressing the GLOBSST01(f) cassette accumulated inulin-type fructose polymers of DP3 through DP7 (FIG. 7). Kernels from transgenic plants expressing both, GLOBSST01(f) and GLOBFFT01(f), cassettes accumulated inulin-type fructose polymers of DP3 through DP10 (FIG. 8). All events that accumulated inulin-type fructose polymers at the somatic embryo stage also accumulated inulin-type fructose polymers at the mature seed stage.

The results shown in FIGS. 3 through 8 indicate two things. First, that fructan may be produced in maize somatic embryos and that these embryos develop into maize plants that produce kernels that make fructan. Second, that following the method of Example 1 a phenotypic kernel trait may be screened at the maize somatic embryo stage and the same trait will be detected in seed from the mature plant. Therefore, the method of Example 1 provides a powerful screening tool for selecting positive transformants at a very early stage. The ability to screen early and obtain the same results as with mature plants results in major labor, financial, and time savings as a substantially less amount of somatic embryos need to be regenerated into plants, as well as less plants need to be maintained for seed production.

Table 1 lists a summary of the results obtained from transforming potato, corn, or mutant corn with SST and FFT, and compares the results according to the tissue analyzed and the expression pattern of the transgene.

TABLE 1
Accumulation of Inulin-type Fructose Polymers in Transgenic Plants

| Plant Species | Tissue | Gene Expression | Inulin (μmol/g f w) |
|---|---|---|---|
| Potato | tuber | tuber | 37.43¹ |
| Corn² | Seed | endosperm | 22.39 |
| Corn | seed | embryo | 80.02 |
| Corn | embryo | embryo | 800.2³ |

¹Data obtained from Hellwege et al. (2000) Proc. Natl. Acad. Sci. USA 97:8698-8704.
²Corn obtained from PCT publication No. WO 99/46395 published Sep. 16, 1999.
³This value is based on the fact that the embryo generally comprises 10% of the kernel fresh weight.

Transgenic kernels expressing the GLOBSST01(f) and GLOBFFT01(f) cassettes accumulated up to 80.02 μmol/g fresh weight fructan. Since the corn embryo alone accounts for 10-20% of the total seed weight, fructan accumulation in the germ can be as high as 800 μmol/g fresh weight. Table 1 shows that the inulin amounts observed in transgenic kernels produced by the method described in this invention is substantially higher than that reported for potato tubers (up to 37.43 μmol/g fresh weight). The differences in inulin production may be due to differences in storage reserve composition and underlying physiology as described in the background section of this invention.

### Example 6

### Construction of Chimeric Vectors for Embryo-Targeted Expression of the Guayule 1-SST and 1-FFT in transgenic Glycine max

Vectors designed for the embryo-specific expression in soybean of guayule (Parthenium argentatum) sucrose:sucrose fructosyltransferase (1-SST) and fructan:fructan fructosyl transferase (1-FFT) were assembled. Vectors pJMS02, pRM02 were designed to express guayule SST, vectors pJMS01 and pRM03 were developed to express guayule FFT, and vectors pRM01 and pRM04 were intended to express both, guayule SST and guayule FFT.

Identification of cDNA Clones Encoding Guayule SST and FFT

Using a guayule (Parthenium argentatum) stem bark library cDNA clones encoding guayule SST and FFT were identified by conducting BLAST (Basic Local Alignment Search Tool; Altschul et al. (1993) J. Mol. Biol. 215:403-410) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

The BLASTX search using the sequences from clone epb3c.pk007.n11 revealed similarity of the polypeptides encoded by the cDNAs to 1-SST from Helianthus tuberosus (NCBI General Identifier No. 3367711) with a pLog higher than 180.00. The BLASTX search using the sequences from clone epb3c.pk007.j9 revealed similarity of the polypeptides encoded by the cDNAs to 1-FFT from Helianthus tuberosus (NCBI General Identifier No. 3367690) with a pLog higher than 180.00.

Amplification of Polynucleotides Encoding Guayule 1-SST or 1-FFT

Polynucleotide fragments encoding the guayule 1-SST or 1-FFT in clones epb3c.pk007.n11 and epb3c.pk007.j9 were amplified by standard PCR methods using Pfu Turbo DNA polymerase (Stratagene, La Jolla, Calif.) and the following primer sets. The oligonucleotide primers were designed to add Not I restriction endonuclease sites at each end of the 1-SST and 1-FFT polynucleotide fragments. Amplification of the cDNA insert in clone epb3c.pk007.n11 was accomplished using oligonucleotide primers SST-3 (shown in SEQ ID NO:8) and SST-4 (shown in SEQ ID NO:9). The resulting polynucleotide encodes an entire guayule 1-SST including secretory and vacuolar targeting signals and its sequence is shown in SEQ ID NO:10.

(SEQ ID NO:8)

5′-AAGCTTGCGGCCGCGCCATGGCTTCMTCHACCACC-3′

(SEQ ID NO: 9)

5′-AAGCTTCTCGAGGCGGCCGCTCAAGAAGTCCACCCAGTAAC-3′

Amplification of the polynucleotide encoding the guayule 1-FFT in clone epb3c.pk007.j9 was performed using the oligonucleotide primers FFT-3 (shown in SEQ ID NO:11) and FFT-4 (shown in SEQ ID NO:12). The resulting polynucleotide fragment encodes an entire guayule 1-FFT including secretory and vacuolar targeting signals and its sequence is shown in SEQ ID NO:13.

| | |
|---|---|
| FFT-3: 5′-AAGCTTGCGGCCGCACCATGGCAACCCCTGAACAACCC-3′ | (SEQ ID NO:11) |
| FFT-4: 5′-AAGCTTCTCGAGGCGGCCGCCTAATTAAACTCGTATTGATG-3′ | (SEQ ID NO:12) |

Assembly of Vectors for the Expression of Guayule 1-SST and 1-FFT

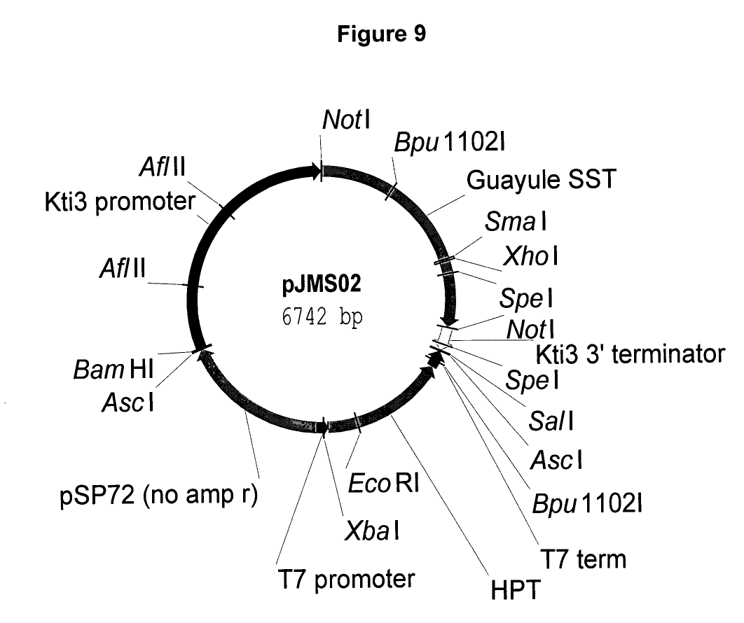

Preparation of pJMS02: The polynucleotide product obtained from amplification of clone epb3c.pk007.n 11 encoding guayule 1-SST was digested with Not I and assembled into vector pJMS02 (shown in FIG. 9) by the following steps. First, the commercially-available plasmid pSP72 (Promega Biotech, Madison, Wis.) was modified to create plasmid pSP72a. Plasmid pSP72 consisted of deletion of the fragment corresponding to the beta lactamase coding region (nucleotides 1135 through 1995), insertion of a polynucleotide fragment comprising the E. coli RNA polymerase T7 promoter operably linked to a polynucleotide encoding HPT the E. coli RNA polymerase T7 promoter and transcription termination, and inserting polynucleotide fragments for the KTi3 promoter and KTi3 transcription termination region. HPT and its function under the control of a bacterial promoter is explained in Example 2. The KTi3 promoter and 3′ transcription terminator region have been described by Jofuku et al. [(1989) Plant Cell 1:1079-1093]. The KTI3 promoter directs strong embryo-specific expression of transgenes. Then, the isolated DNA fragment containing the guayule SST was inserted into Not 1-digested plasmid pSP72a to obtain plasmid pJMS02 the sequence of which is shown in SEQ ID NO:14.

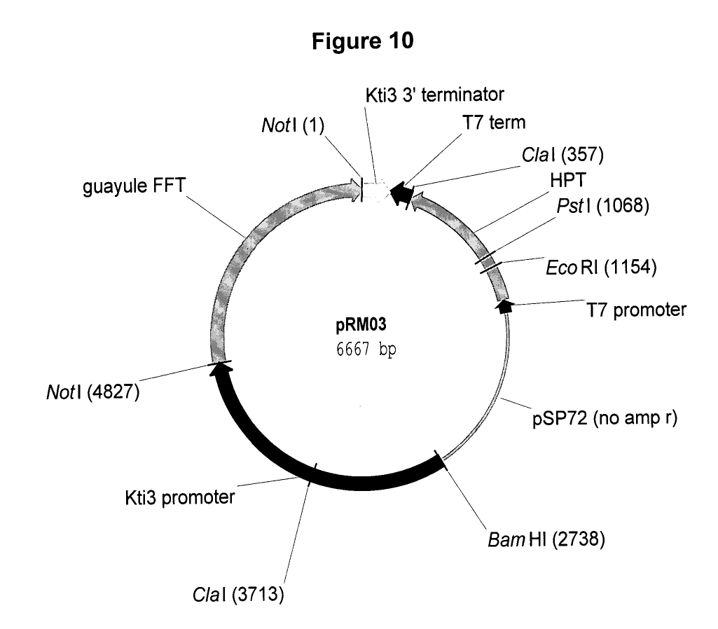

Preparation of pRM03: Vector pRM03 comprises nucleotides encoding guayule FFT under the control of a KTi3 promoter and termination signals and nucleotides encoding HPT under control of the T7 promoter and termination signals. To produce vector pRM03 the polynucleotide product encoding guayule 1-FFT obtained from amplification of clone epb3c.pk007.j9 was digested with Not I and used to replace the 1-SST polynucleotide fragment from clone pJMS02 to create plasmid pRM03. The 1-SST polynucleotide fragment had been removed from pJMS02 by digestion with Not I. Vector pRM03 is depicted in FIG. 10 and contains two expression cassettes. One cassette contains the KTi3 promoter directing the expression of the guayule 1-FFT (including secretory and vacuolar targeting signals) followed by the KTi3 transcription terminator. Another cassette comprises the E. coli RNA polymerase T7 promoter directing the expression of HPT followed by the T7 transcription terminator. The polynucleotide sequence of vector pRM03 is shown in SEQ ID NO:15.

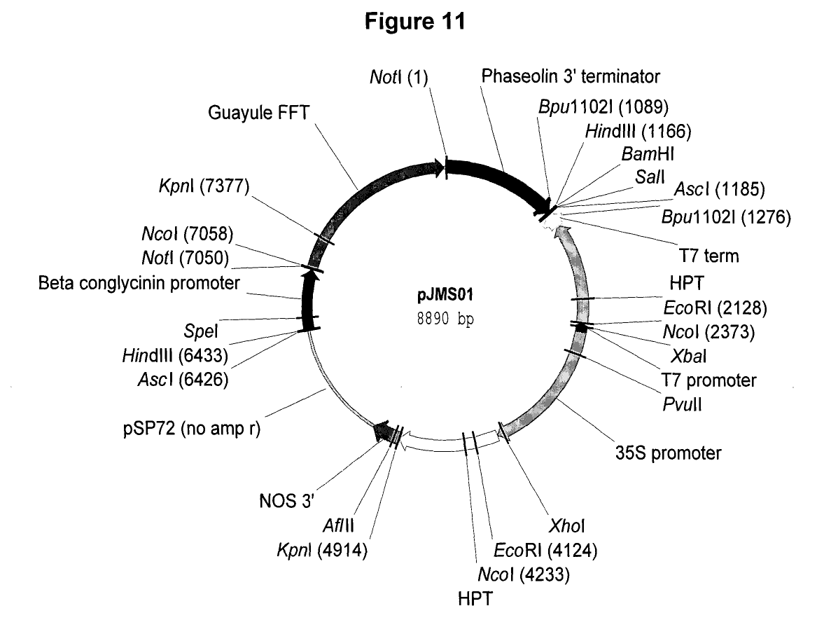

Preparation of pJMS11: Vector pJMS01 comprises nucleotides encoding guayule 1-FFT under the control of the beta conglycinin promoter and phaseolin terminator. This vector also comprises nucleotides encoding HPT under the control of the T7 promoter and termination signals and the 35S promoter and Nos 3′ terminator. To produce vector pJMS01 the polynucleotide product encoding guayule 1-FFT obtained from amplification of clone epb3c.pk007.j9 was digested with Not I and inserted into Not I-digested soybean expression vector pKS123 to generate the vector pJMS01 (depicted in FIG. 11). Vector pKS123 contains a cassette for the expression of HPT under the CaMV 35S promoter and nos 3′ end and a cassette comprising a β-conglycinin promoter and the phaseolin 3′ transcription terminator separated by a Not I restriction endonuclease site. To prepare vector pKS123, a cassette comprising a CaMV 35S promoter directing the expression of HPT followed by a nos 3′ end, and flanked on either side with Sal I sites was introduced into vector pKS17 (described in Example 2). This modified vector pKS17 was digested with Xho I and Sal I followed by treatment with mung bean nuclease (to make blunt the resulting ends) and a linker primer introduced. The sequence of this linker primer is shown in SEQ ID NO:16 and contains, in a 5′ to 3′ orientation processing sites for the restriction endonucleases Asc I, Hind III, Bam HI, Sal I, and Asc I.

5′-GGCGCGCCAAGCTTGGATCCGTCGACGGCGCGCC-3′ (SEQ ID NO:16)

After ligation the modified vector was digested with Hind III and the cassette comprising the β-conglycinin promoter and phaseolin 3′ transcription terminator separated by a Not I restriction endonuclease site was added to form vector KS123. The CaMV 35S promoter has been described by Odell et al. ((1985) Nature 313:810-812; and Hull et al. (1987) Virology 86:482-493). The nopaline synthase transcription terminator has been described by Depicker et. al. ((1982) J. Appl. Genet. 1:561-574). The β-conglycinin promoter fragment is an allele of the β-conglycinin promoter published by Doyle et al. ((1986) J. Biol. Chem. 261:9228-9238). The 1175 base pair phaseolin transcription terminator has been described by Doyle et al. ((1986) J. Biol. Chem. 261:9228-9238; and Slightom et al. (1983) Proc. Natl. Acad. Sci. USA 80:1897-1901). The amplified guayule 1-FFT was digested with Not I and introduced into Not 1-digested vector pKS123 to form plasmid pJMS01. Plasmid pJMS01 contains then, β-conglycinin promoter operably linked to the guayule 1-FFT, operably linked to the phaseolin transcription terminator. Plasmid pJMS01 also contains fragments for the expression of HPT in bacteria (under the control of the T7 promoter) and in eukaryotic systems (under the control of the CaMV 35S promoter). These two cassettes allow for selection of transformed cells in bacterial and plant systems in the presence of hygromycin. The nucleotide sequence of plasmid pJMS01 is shown in SEQ ID NO:17.

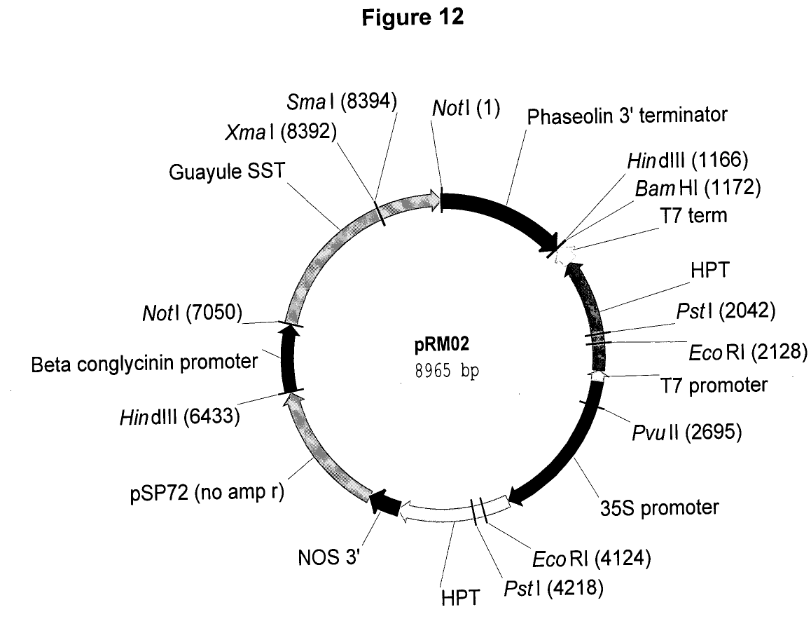

Preparation of pRM02: Vector pRM02 (shown in FIG. 12) comprises nucleotides encoding guayule SST under the control of the beta conglycinin promoter and phaseolin 3′ terminator and nucleotides encoding HPT under was prepared by replacing the polynucleotide fragment encoding guayule 1-FFT from plasmid pJMS01 with the polynucleotide fragment encoding guayule 1-SST in plasmid pJMS02. Removal of the guayule 1-FFT and 1-SST fragments was accomplished by digestion with Not I. Plasmid pRM02 is depicted in FIG. 12 and contains the β-conglycinin promoter operably linked to the guayule 1-SST, operably linked to the phaseolin transcription terminator. Plasmid pRM02 also contains cassettes for the expression of HpT in bacterial and plant systems useful for selection of transformed cells. The nucleotide sequence of plasmid pRM02 is shown in SEQ ID NO:18.

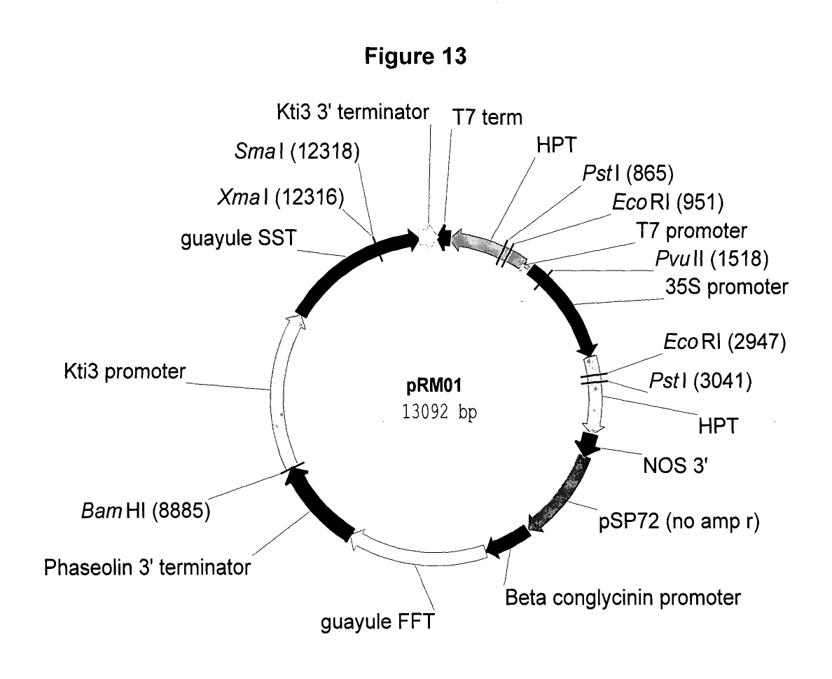

Preparation of pRM01: Vector pRM01 comprises nucleotides encoding guayle 1-SST under control of the KTi3 promoter and termination signals and nucleotides encoding guayule 1-FFT under control of the beta conglycinin promoter and phaseolin terminator. Vector pRM01 also comprises nucleotides encoding HPT under the control of the 35S promoter and nos terminator and T7 promoter and terminator. Plasmid pRM01 (shown in FIG. 13) was constructed by removing the polynucleotide fragment containing the KTi3 promoter/guayule 1-SST coding region/KTi transcription terminator cassette from plasmid pJMS02 and transferring it to plasmid pJMS01. The KTi3 promoter/guayule 1-SST coding region/KTi transcription terminator cassette was removed from plasmid pJMS02 by digestion with Bam HI, which cuts immediately upstream of the KTi3 promoter, and Sal I, which cuts in the KTi3 transcription terminator region. Plasmid pJMS01 was digested with Bam HI and Sal I, which cut between the T7 and phaseolin terminator regions. Plasmid pRM01 contains the polynucleotide encoding guayule 1-SST under the control of the KTi3 promoter and transcription terminator and the polynucleotide encoding guayule 1-FFT under the control of the phaseolin promoter and transcription terminator. Plasmid pRM01 also contains cassettes for the expression of HPT in bacterial and plant systems useful for selection of transformed cells. The polynucleotide sequence of plasmid pRM01 is shown in SEQ ID NO:19.

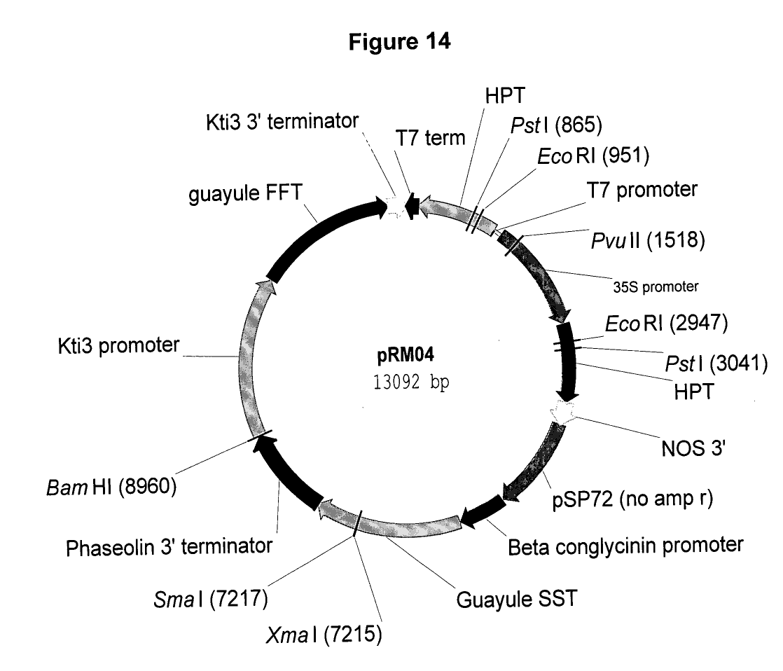

Preparation of pRM04: Vector pRM04 comprises nucleotides encoding guayule 1-SST under the control of the beta conglycinin promoter and phaseolin terminator and nucleotides encoding guayule 1-FFT under control of the KT13 promoter and terminator. Vector pRM04 also comprises nucleotides encoding HPT under the control of the T7 promoter and termination signals and the 35S promoter and nos terminator. Plasmid pRM04 (shown in FIG. 14) was constructed by transferring the KTi3 promoter/guayule 1-FFT coding region/KTi3 transcription terminator cassette from pasmid pRM03 to plasmid pRM02. Digestion with Bam HI and Sal I was used to remove the 1-FFT expression cassette from plasmid pRM03 and insert it between the T7 and the phaseolin transcription terminators. Plasmid pRM04 contains the polynucleotide encoding guayule 1-SST under control of the beta conglycinin promoter and phaseolin transcription terminator and the polynucleotide encoding guayule 1-FFT under control of the KTi3 promoter and transcription terminator. Plasmid pRM04 also contains cassettes for the expression of HPT in bacterial and plant systems useful for selection of transformed cells. The polynucleotide sequence of plasmid pRM04 is shown in SEQ ID NO:20.

### Example 7

### Transformation of Soybean Somatic Embryos with Guayule SST and FFT Expression Vectors

To study the possibility of producing inulin in soybeans, soybean somatic embryos were transformed with the seed-specific expression vectors expressing the guayule 1-SST and 1-FFT. Soybean somatic embryos were transformed with plasmids pJMS01 and pJMS02, plasmids pRM02 and pRM03, plasmid pRM01, or plasmid pRM04 by the method of particle gun bombardment (Klein, T. M. et al. (1987) Nature (London) 327:70-73; U.S. Pat. No. 4,945,050).

Soybean somatic embryos from the Jack cultivar were induced as follows. Cotyledons (3 mm in length) were dissected from surface sterilized, immature seeds and were cultured for an additional 6-10 weeks in the light at 26° C. on a Murashige and Skoog media containing 7 g/L agar and supplemented with 10 mg/mL 2,4-D. Globular stage somatic embryos, which produced secondary embryos, were then excised and placed into flasks containing liquid MS medium supplemented with 2,4-D (10 mg/mL) and cultured in the light on a rotary shaker. After repeated selection for clusters of somatic embryos that multiplied as early, globular staged embryos, the soybean embryogenic suspension cultures were maintained in 35 mL liquid media on a rotary shaker, 150 rpm, at 26° C. with fluorescent lights on a 16:8 hour day/night schedule. Cultures were subcultured every two weeks by inoculating approximately 35 mg of tissue into 35 mL of liquid medium.

Soybean embryogenic suspension cultures were then transformed by the method of particle gun bombardment (Klein, T. M., et al. (1987) Nature (London) 327:70-73, U.S. Pat. No. 4,945,050) using a DuPont Biolistic™ PDS1000/HE instrument (helium retrofit). To 50 μL of a 60 mg/μL 1 mm gold particle suspension were added (in order): 5 μL of 1 mg/μL DNA (pJMS01 plus pJMS02, pRM02 plus pRM03, pRM01, or pRM04), 20 μl of 0.1 M spermidine, and 50 μL of 2.5 M CaCl₂. The particle preparation was then agitated for three minutes, spun in a microfuge for 10 seconds and the supernatant removed. The DNA-coated particles were then washed once in 400 μL 70% ethanol and resuspended in 40 μL of anhydrous ethanol. The DNA/particle suspension was sonicated three times for one second each. Five μL of the DNA-coated gold particles was then loaded on each macro carrier disk.

Approximately 300-400 mg of a two-week-old suspension culture was placed in an empty 60×15 mm Petri dish and the residual liquid removed from the tissue with a pipette. For each transformation experiment, approximately 5 to 10 plates of tissue were bombarded. Membrane rupture pressure was set at 1100 psi and the chamber was evacuated to a vacuum of 28 inches mercury. The tissue was placed approximately 3.5 inches away from the retaining screen and bombarded three times. Following bombardment, the tissue was divided in half and placed back into liquid and cultured as described above.

Five to seven days post bombardment, the liquid media was exchanged with fresh media, and eleven to twelve days post bombardment with fresh media containing 50 mg/mL hygromycin. This selective media was refreshed weekly. Seven to eight weeks post bombardment, green, transformed tissue was observed growing from untransformed, necrotic embryogenic clusters. Isolated green tissue was removed and inoculated into individual flasks to generate new, clonally propagated, transformed embryogenic suspension cultures. Each new line was treated as an independent transformation event. These suspensions were then subcultured and maintained as clusters of immature embryos. Storage products produced by immature embryos at this stage are similar in composition to storage products produced by zygotic embryos at a similar stage of development (see PCT Publication No. WO 94/11516, published May 26, 1994).

### Example 8

### Carbohydrate Analysis of Transgenic Soybean Somatic Embryos

The carbohydrate profiles of soybean somatic embryos resulting from transformation with the vectors containing cassettes for embryo-specific expression of guayule 1-SST and 1-FFT were determined following the protocol shown in Example 5.

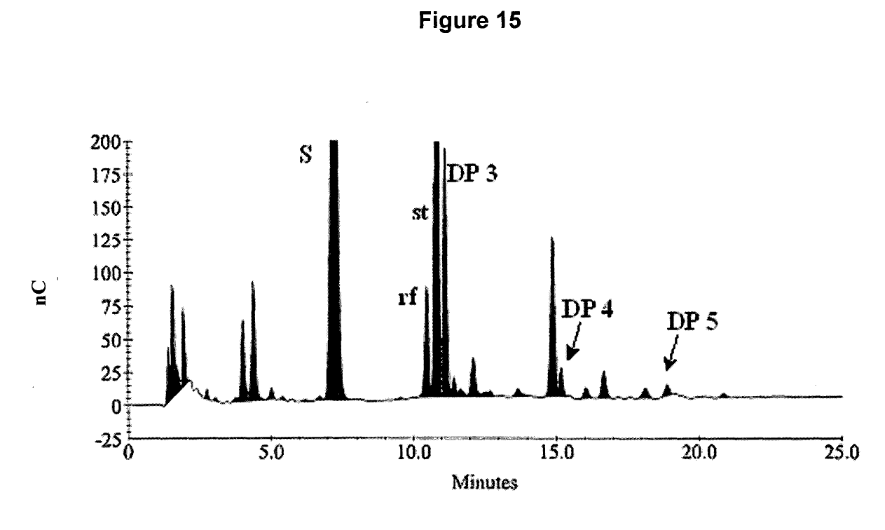

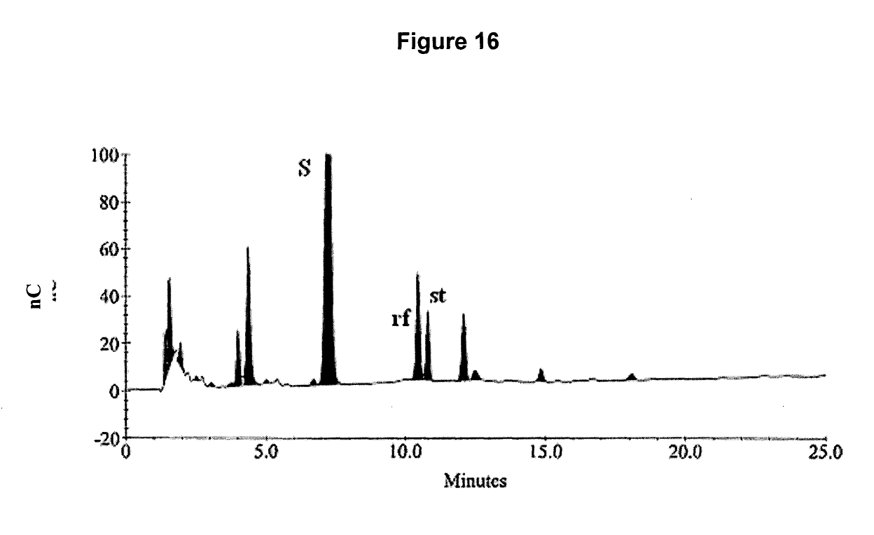

FIG. 15 shows the carbohydrate profile resulting from HPAE/PAD analysis of transgenic soybean somatic embryos expressing guayule 1-SST and 1-FFT. These embryos express the guayule 1-SST and 1-FFT from vectors pRM02 and pRM03, and accumulate inulin of DP3 to DP5. These compounds are absent in soybean somatic embryos that were not transformed with the expression cassettes (negative controls) but were generated using the same procedures as described in Example 7 (FIG. 16). Similar profiles were observed for somatic embryos expressing both, guayule 1-SST and 1-FFT, resulting from transformations using pRM01 or RM04.

The data presented here shows, for the first time, that transgenic soybean embryos are capable of producing inulin when expressing 1-SST and 1-FFT. The polynucleotides used in the present application to transform soybean encode 1-SST and 1-FFT enzymes that are normally expressed in the bark tissue of the rubber tree. The Jerusalem artichoke SST and FFT, used here to transform corn, are expressed in the tuber.

Corn kernels accumulate longer inulin than maize somatic embryos, as shown in Example 5, it is expected that soybean seeds will produce more and longer inulin than the somatic embryos.

In the examples above, it is demonstrated that embryo-specific expression of 1-SST and 1-FFT results in inulin accumulation in plants that normally do not accumulate this carbohydrate. Furthermore, that embryo-specific expression of Jerusalem artichoke 1-SST and 1-FFT results in larger accumulation of inulin than the previously shown endosperm-specific expression of the same 1-SST and 1-FFT. It is also demonstrated that embryo-specific expression of guayule 1-SST and 1-FFT in soybean somatic embryos results production of inulin. Considering that the guayule enzymes are normally expressed in the bark tissue of the rubber tree, expression in the embryo was unexpected. The examples above also show that analysis of somatic embryos for a given trait allows an accurate and quick method for determining successful transformation events.

### Example 9

### Carbohydrate Analyses of Transgenic Soybean Embryos and Seeds

Carbohydrate profiles of dried-down transgenic somatic soybean embryos and of transgenic soybean seeds expressing guayule 1-SST and 1-FFT from plasmid pRM01 were obtained. Somatic soybean embryos were transformed with vector pRM01 as described in Example 7. Immature transgenic somatic soybean embryos expressing guayule 1-SST and 1-FFT were selected as in Example 1. The somatic soybean embryos were dried-down to mimic the last stages of soybean seed development. Dried-down embryos are capable of producing plants when transferred to soil or soil-less media.

Analysis of Dried Transgenic Soybean Embryos

Immature transgenic somatic soybean embryos expressing guayule 1-SST and 1-FFT were dried-down to mimic the last stages of soybean development especially the seed dry down phase. To dry-down, somatic embryos were transferred to an empty petri dish, covered, and put in a second petri dish containin modified MS medium (described in Example 1) and allowed to dry for 2 to 5 days. The carbohydrate profile of dried-down individual somatic embryos was determined essentially as described in Example 5, with minor modifications. The analysis was modified by using a CarboPac PA100 anion exchange column and guard column which enabled inulin detection of DP>15.

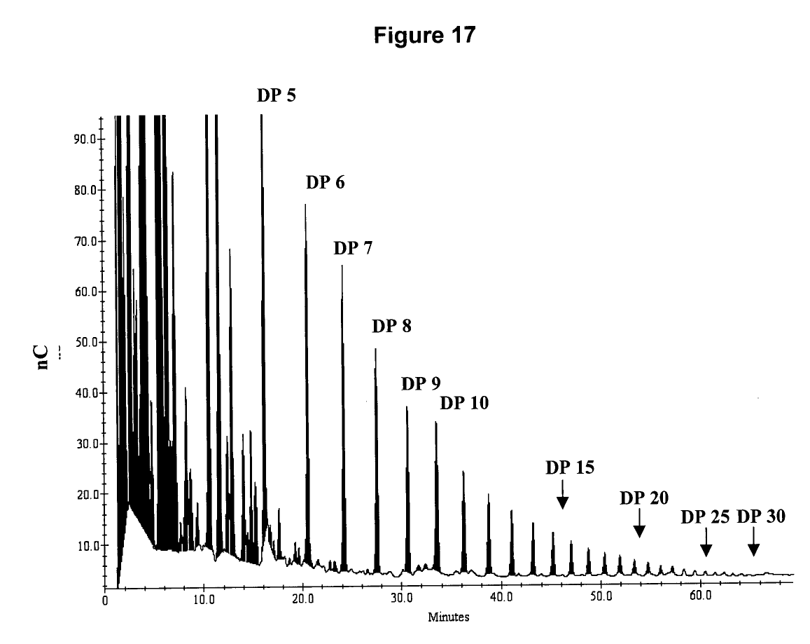

A typical carbohydrate profile obtained for dried-down soybean embryos expressing guayule 1-SST and 1-FFT from vector pRM01 is shown in FIG. 17. This carbohydrate profile clearly shows that inulin, of DP 3 to at least DP30, is detected in dried-down soybean somatic embryos. No inulin is detectable in dried-down soybean embryos that have gone through the same process but do not express 1-SST or 1-FFT. This is the first time where soybean somatic embryos are shown to produce fructans. These data support the fact that the guayule 1-SST and 1-FFT are expressed and active in soybean.

Carbohydrate Analysis of Mature Seeds from Transgenic Soybean

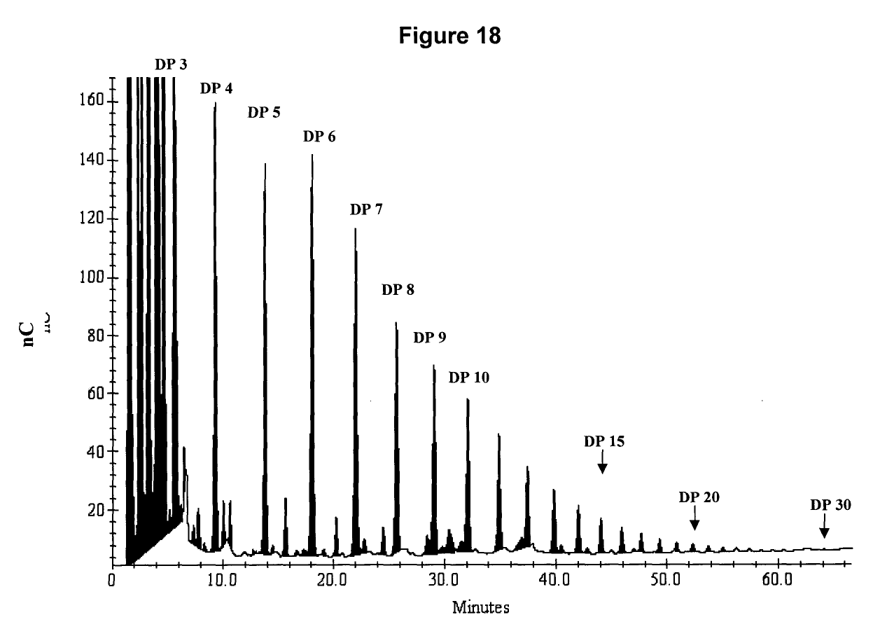

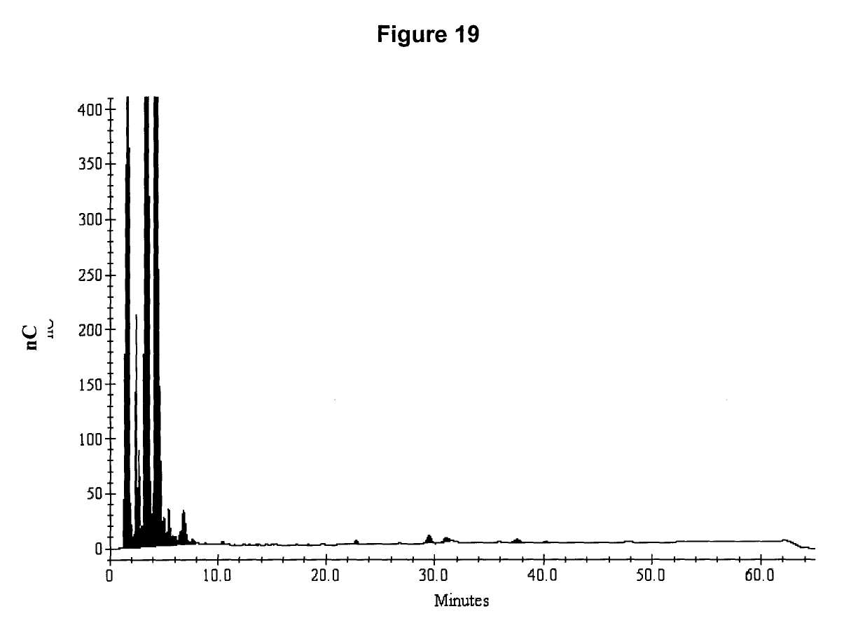

Somatic embryos dried-down as described above were transferred to a soil-less mixture to enable their development into plants. Transgenic plants from all transformation events were allowed to set seed and individual mature seeds were obtained. The carbohydrate profile of mature soybean seeds was determined as described in Example 5. A typical carbohydrate profile of individual seeds from transgenic soybean plants expressing intact copies of the guayule 1-SST and 1-FFT from vector pRM01 is shown in FIG. 18. This Figure shows that transgenic soybean seeds expressing the guayule 1-SST and 1-FFT accumulated inulin-type fructose polymers of DP 3 through at least DP 30. It is possible that accumulation of inulins of DP larger than 30 may still occur but their levels fall below current detection limits. The carbohydrate profile resulting from HPAE/PAD analysis of chips from seeds from transgenic soybean plants not expressing guayule 1-SST and 1-FFT is shown in FIG. 19 where no fructans are detected.

The results presented above show that fructans may be produced in soybean somatic embryos and that these embryos are capable of developing into soybean plants that produce seeds that make fructans. Furthermore they also show that, as with corn, a phenotypic seed trait may be identified at the soybean somatic embryo stage and the same trait will be present in seed from the mature plants.

What is claimed is:

1. A plant comprising at least one recombinant DNA molecule comprising an embryo specific promoter operably linked to at least a portion of at least one coding sequence for a plant fructosyltransferase, operably linked to a vacuole targeting sequence, said molecule sufficient to express a protein capable of producing fructan having a degree of polymerization of at least three, in an embryo of said plant, or any progeny thereof, wherein said progeny comprise said molecule.

2. The plant of claim 1 wherein said fructan is inulin.

3. The plant of claim 1 wherein said fructosyltransferase is selected from the group consisting of sucrose:sucrose fructosyltransferase, and the combination of sucrose:sucrose fructosyltransferase and fructose:fructose fructosyltransferase.

4. The plant of claim 1 wherein said plant is a cereal.

5. The plant of claim 1 wherein said plant is corn.

6. The plant of claim 1 wherein said plant is soybean.

7. The plant of claim 1 wherein said coding sequence for fructosyltransferase is selected from the group consisting of a monocot and a dicot.

8. The plant of claim 1 wherein said at least one fructosyltransferase comprises a sucrose:sucrose fructosyltransferase.

9. The plant of claim 1 wherein said at least one fructosyltransferase comprises a first fructosyltransferase and a second fructosyltransferase, wherein said first fructosyltransferase comprises sucrose:sucrose fructosyltransferase and said second fructosyltransferase comprises fructose:fructose fructosyltransferase.

10. The plant of claim 1 wherein said at least one DNA molecule comprises a first DNA molecule and a second DNA molecule, said first DNA molecule comprises sucrose:sucrose fructosyltransferase and said second DNA molecule comprises fructose:fructose fructosyltransferase.

11. The plant of claim 1 wherein said at least one DNA molecule comprises sucrose:sucrose fructosyltransferase and fructose:fructose fructosyltransferase.

12. Fructan produced by the enzyme encoded by the coding sequence in the recombinant DNA molecule from the plant of claim 1.

13. A recombinant DNA molecule comprising an embryo specific promoter operably linked to at least a portion of at least one coding sequence for a fructosyltransferase, operably linked to a vacuole targeting sequence, said molecule sufficient to express a protein capable of producing fructan in an embryo cell.

14. The recombinant DNA molecule of claim 13 wherein said fructan is inulin.

15. The recombinant DNA molecule of claim 13 wherein said fructosyl-transferase is selected from the group consisting of sucrose:sucrose fructosyltransferase, and the combination of sucrose:sucrose fructosyltransferase and fructose:fructose fructosyltransferase.

16. A method of producing fructan in a plant comprising:
a) constructing at least one recombinant DNA molecule comprising an embryo specific promoter operably linked to a vacuole targeting sequence operably linked to at least a portion of at least one coding sequence for a fructosyltransferase,
b) transforming a plant cell with said construct,
c) regenerating said plant to produce seed,
d) harvesting seed from said plant of step c, and
e) extracting fructan from seed of step d.

17. The method according to claim 16, wherein said fructan is inulin.

18. A method of screening transgenic maize tissue expressing embryo targeted genes comprising:
a) preparing Type-II maize callus for transformation,
b) transforming callus,
c) selecting transgenic callus lines,
d) regenerating transgenic somatic embryos, and
e) propagating transgenic somatic embryos for both plant production and early trait analyses.

19. A foodstuff comprising fructan obtained from a plant comprising at least one recombinant DNA molecule comprising an embryo specific promoter operably linked to a vacuole targeting sequence operably linked to at least a portion of at least one coding sequence for a fructosyltransferase, said molecule sufficient to express a protein capable of producing fructan of at least DP3 in a grain of said plant, or any progeny thereof, wherein said progeny comprise said molecule.

20. A foodstuff comprising inulin obtained from a plant comprising at least one recombinant DNA molecule comprising an embryo specific promoter operably linked to a vacuole targeting sequence operably linked to at least a portion of at least one coding sequence for a fructosyltransferase, said molecule sufficient to express a protein capable of producing fructan of at least DP3 in a grain of said plant, or any progeny thereof, wherein said progeny comprise said molecule.

21. An industrial product comprising fructan obtained from a plant comprising at least one recombinant DNA molecule comprising an embryo specific promoter operably linked to a vacuole targeting sequence operably linked to at least a portion of at least one coding sequence for a fructosyltransferase, said molecule sufficient to express a protein capable of producing fructan of at least DP3 in a grain of said plant, or any progeny thereof, wherein said progeny comprise said molecule.

22. The industrial product of claim 21 selected from the group consisting of a hydrocolloid, a bleach activator, a dispersing agent, a glue, and a biodegradable complexing agent.

23. Grain of the plant of claim 1.

24. Grain of the plant of claims 5 or 6.

25. A foodstuff comprising grain of the plant of claim 1.

26. A foodstuff comprising grain of the plant of claims 5 or 6.
